(19) United States
(12) Patent Application Publication
(10) Pub. No.: US 2023/0233699 A1
(43) Pub. Date: Jul. 27, 2023

(54) OLIGONUCLEOTIDE CONJUGATES AND PREPARATION AND APPLICATIONS THEREOF

(71) Applicant: ACADEMIA SINICA, Taipei City (TW)

(72) Inventors: Andrew H.-J. WANG, San Diego, CA (US); Cheng-Chung LEE, Taichung City (TW); Nai-Shu HSU, Taipei City (TW); Wen-Chih KUO, New Taipei City (TW)

(73) Assignee: ACADEMIA SINICA, Taipei City (TW)

(21) Appl. No.: 17/918,947

(22) PCT Filed: Apr. 15, 2021
(86) PCT No.: PCT/US21/27403

§ 371 Date: Oct. 14, 2022

Related U.S. Application Data
(60) Provisional application No. 63/010,167, filed on Apr. 15, 2020.

(51) Int. Cl. A61K 47/68; A61K 47/58; A61K 47/54

(52) U.S. Cl. CPC A61K 47/6803; A61K 47/58; A61K 47/6889; A61K 47/549

(57) ABSTRACT

The present invention relates to oligonucleotide conjugates and preparation and applications thereof. In particular, the present invention relates to an oligonucleotide conjugated to a biomolecule (e.g. an antibody) and/or an agent of interest (e.g. a drug). In certain embodiments, the oligonucleotide of the present invention is a hybridized complex of a single strand oligonucleotide carrying a biomolecule and a complementary strand oligonucleotide bearing an agent of interest where the hybridized nucleotide segment acts as a linker to link the biomolecule and the agent of interest in one molecule.

### RELATED APPLICATIONS

This application claims the benefit of U.S. provisional application No. 63/010,167, filed Apr. 15, 2020 under 35 U.S.C. § 119, the entire content of which is incorporated herein by reference.

### TECHNOLOGY FIELD

The present invention relates to oligonucleotide conjugates and preparation and applications thereof. In particular, the present invention relates to an oligonucleotide conjugated to a biomolecule (e.g. an antibody) and/or an agent of interest (e.g. a drug). In certain embodiments, the oligonucleotide of the present invention is a hybridized complex of a single strand oligonucleotide carrying a biomolecule and a complementary strand oligonucleotide bearing an agent of interest where the hybridized nucleotide segment acts as a linker to link the biomolecule and the agent of interest in one molecule.

### BACKGROUND OF THE INVENTION

Antibody-drug conjugates (ADCs) are an emerging class of cancer therapeutics, allowing for specific delivery of highly potent drugs to malignant cells. However, to this date, there have only been three marketed ADCs, which are Adcetris (2011), Kadcyla (2013), and Besponsa (2017), demonstrating that, despite the simplicity of its concept, the development of ADCs remains a major challenge.¹

In an ADC, a cytotoxic payload is attached to an antibody via a linker, which is of paramount importance to the success of ADCs.^{2,3} Ideally, a linker should remain stable in the plasma during circulation, but rapidly release its drug load upon internalization into target cancer cells.³ Hydrophobicity is another crucial issue of linker design. ADCs with hydrophobic linkers tend to form aggregates, which may result in problems such as hepatotoxicity due to altered pharmacokinetic properties, as well as immunogenicity in the bloodstream.^{3,4} Moreover, drugs attached to hydrophobic linkers are better substrates of multidrug resistance (MDR) transporters and lose their efficacies against MDR-expressing cell lines.³ In order to address these difficulties, recent attempts to incorporate charged residues, e.g. sulfonate or pyrophosphate, into linkers are met with promising results, suggesting that hydrophilic linkers are highly desirable for new ADC formats.^{5,6}

Antibody-oligonucleotide conjugates (ADCs) are bifunctional molecules that have seen increasing applications in various fields, including therapy, diagnosis, and imaging.⁷ In a pioneering work by Cantor et al, the ability of oligonucleotides to be amplified exponentially by polymerase chain reaction (PCR) was elegantly combined with the high antigen-binding specificity of antibodies, thereby enhancing the sensitivity of traditional immunosorbent assays by several orders of magnitude.⁸ This technique, termed immuno-PCR, has been actively explored ever since.⁹⁻¹¹ AOCs have been applied to radiotherapy of cancer as well. Traditionally, radioactive elements were brought to the proximity of tumors through direct conjugation with antibodies. Normal tissues, however, suffer from significant exposure to radiation in the process due to the slow clearance rate and poor tumor penetration kinetics of antibodies.¹² Works by Constant et al opened up the possibility to first saturate tumors with AOCs, followed by administration of complementary strands carrying radioactive elements that would hybridize to the tumor-bound AOCs.^{13,14} This two-step, or pre-targeting approach, has the advantage of minimal normal tissue exposure thanks to the much faster clearance of oligonucleotides compared to antibodies.¹² Preclinical results of AOCs applied in radiotherapy of cancer are encouraging.^{5,16} When constructed with internalizing antibodies, AOCs can also be used to deliver functional nucleic acids into the cells, such as anti-sense oligonucleotides or small-interfering RNAs (siRNAs).^{17, 18}

There is a need to provide a simple and straight-forward approach to efficiently and rapidly produce oligonucleotide conjugates of desired functions.

### SUMMARY OF THE INVENTION

The present disclosure is based, at least in part, on the development of a flexible and modular linker strategy for making an oligonucleotide conjugate based on oligonucleotide strand-pairing. The present invention accordingly provides oligonucleotide conjugates and preparation and applications thereof.

In one aspect, the present invention provides an oligonucleotide conjugate which comprises

(i) a first oligonucleotide conjugate comprising a first single strand oligonucleotide conjugated to a biomolecule, wherein the first single strand oligonucleotide comprises a first nucleotide sequence; and/or

(ii) a second oligonucleotide conjugate comprising a second single strand oligonucleotide conjugated to an agent, wherein the second single strand oligonucleotide comprises a second nucleotide sequence being complementary to the first nucleotide sequence;

wherein the first and second oligonucleotide conjugates form a double-strand oligonucleotide conjugate which comprises a hybridized oligonucleotide bridge region between the first nucleotide sequence and the second nucleotide sequence, whereby the biomolecule and the agent are linked together in the double-strand oligonucleotide conjugate.

In some embodiments, the first and second nucleotide sequences individually comprises a GC rich sequence.

In some embodiments, the first nucleotide sequence and the second nucleotide sequence individually has substantially no secondary structure.

In some embodiments, the first and second nucleotide sequences have a melting temperature (Tm) of at least 38° C. (e.g. 38° C.−100° C.). In some instances, the Tm is about 40° C.−70° C., such as 41° C.−69° C., 43° C.−67° C., 45° C.−65° C., 47° C.−63° C., 49° C.−60° C., 51° C.−59° C. or 53° C.−57° C.

In some embodiments, the first single strand oligonucleotide is conjugated at 3′-end to the targeting biomolecule, and/or the second single strand oligonucleotide is conjugated at 3′-end to the agent.

In some embodiments, the first single strand oligonucleotide is conjugated at 5′-end to the targeting biomolecule, and/or the second single strand oligonucleotide is conjugated at 5′-end to the agent.

In some embodiments, the first single strand oligonucleotide, the second single strand oligonucleotide, or both are DNAs, RNAs, or hybrids thereof.

In some embodiments, the first single strand oligonucleotide, the single strand second oligonucleotide, or both comprise at least one modified nucleotide residue.

In some embodiments, the GC rich sequence comprises

the nucleotide sequence 5′-SSWSSWSWSSSWWSSWSS-3′ as set forth in SEQ ID NO:1, wherein each S is independently selected from G or C and each W is independently selected from A or T; or

the nucleotide sequence 5′-SSWSSWWSSSWSWSSWSS-3′ as set forth in SEQ ID NO:2, wherein each S is independently selected from G or C and each W is independently selected from A or T.

In particular embodiments, the GC rich sequence comprises

the nucleotide sequence 5′-GGWCCWGWCCGWWGGWCC-3′ as set forth in SEQ ID NO: 3 wherein each W is independently selected from A or T; or

the nucleotide sequence 5′-GGWCCWWCGGWCWGGWCC-3′ as set forth in SEQ ID NO: 4, wherein each W is independently selected from A or T.

In certain examples, the GC rich sequence comprises

the nucleotide sequence
(SEQ ID NO: 5)
5′-GGACCAGACCGAAGGACC-3′;
or
the nucleotide sequence
(SEQ ID NO: 6)
5′-GGTCCTTCGGTCTGGTCC-3′.

In some embodiments, the (first/second) oligonucleotides, each or both, contain about 12 to 80 nucleotides in length for example, about 15 to about 60 nts, about 15 to about 50 nts, about 15 to about 40 nts, about 15 to about 30 nts, about 15 nts to about 25 nts, or about 15 to about 20 nts. In some examples, the one or both oligonucleotides may contain about 15 to about 25 nts, for example, about 18 nts.

In some embodiments, the biomolecule is a peptide, a polypeptide, a nucleic acid, or a carbohydrate molecule.

In certain embodiments, the biomolecule is a targeting molecule e.g. an antibody.

In some embodiments, the (first) oligonucleotide is conjugated to a biomolecule via a chemical linker. Examples of the chemical linker include but are not limited to a succinimide moiety, a maleimide moiety, a hydrazine moiety, a tyrosine moiety, a hydrazone moiety, an azide moiety, a terminal alkyne moiety, a strained terminal alkyne moiety, or a phosphine moiety.

In some embodiments, the molar ratio between the biomolecule and the first oligonucleotide ranges from 1:1 to 1:6 (e.g. 1:1, 1:2, 1:3, 1:4, 1:5 or 1:6).

In some embodiments, the agent conjugated to the second oligonucleotide is a therapeutic agent or a diagnostic agent.

In certain embodiments, a therapeutic agent is a cytotoxic agent. Examples of the cytotoxic agent include but are not limited to monomethyl auristatin E (MMAE) or mertansine (DM1).

In certain embodiments, a diagnostic agent is a fluorescent moiety, a luminescent moiety or a radioactive moiety.

In another aspect, the present invention provides a method of preparing an oligonucleotide-linked molecule, the method comprising (a) providing a first oligonucleotide conjugate comprising a first oligonucleotide conjugated to a biomolecule, wherein the first oligonucleotide comprises a first nucleotide sequence; (b) providing a second oligonucleotide conjugate comprising a second oligonucleotide conjugated to an agent, wherein the second oligonucleotide comprises a second nucleotide sequence being complementary to the first nucleotide sequence; and (c) incubating the first oligonucleotide conjugate and the second oligonucleotide conjugate under conditions allowing for hybridization between the first oligonucleotide and the second oligonucleotide, thereby producing an oligonucleotide-linked molecule carrying both of the biomolecule and the agent. Optionally, the method may further comprise (d) harvesting the oligonucleotide-linked molecule produced in step (c). Exemplary features of the first oligonucleotide, the first nucleotide sequence, the biomolecule to be conjugated to the first oligonucleotide, the second oligonucleotide, the second nucleotide sequence and the agent to be conjugated to the second oligonucleotide are as described above.

In some embodiments, step (a) in any of the methods disclosed herein can be performed by a process comprising: (al) adding a first functional handle to the 5′ end of the first oligonucleotide to form a reactive first oligonucleotide; and (a2) reacting the reactive first oligonucleotide with the biomolecule to produce the first oligonucleotide conjugate. In some instances, the first functional handle is a maleimide moiety and the biomolecule is a polypeptide comprising a free—SH group, e.g., the polypeptide (e.g. antibody) comprises an internal disulfide bridge and can be treated by a reducing agent to produce the free—SH group. In some instances, step (al) can be performed by reacting the first oligonucleotide with succinimidyl 4-(N-maleimidomethyl)cyclohexane-1-carboxylate.

In some embodiments, step (b) in any of the methods disclosed herein may be performed by a process comprising (1)1) adding a second functional handle to the 5′ end of the second oligonucleotide to produce a reactive second oligonucleotide; and (b2) incubating the reactive second oligonucleotide and the agent in the presence of a cross-linking reagent to produce the agent conjugated with the second oligonucleotide. In some instances, the second functional handle is a —SH group or a —NH₂ group. Alternatively or in addition, the cross-linking agent is succinimidyl 4-(N-maleimidomethyl)cyclohexane-1-carboxylate or 2,2′-dithiodipyridine.

Further, provided herein are methods for treating or diagnosing a disease in a subject in need thereof, the method comprising administering to the subject any of the oligonucleotide conjugates disclosed herein. Also within the scope of the present disclosure are any of the oligonucleotide conjugates or pharmaceutical compositions comprising such for use in treating a suitable target disease or disorder, or for use in manufacturing a medicament for treatment of the target disease or disorder.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a schematic illustration showing the design and preparation of an exemplary antibody-drug conjugate (ADC) based on oligonucleotide strand-pairing. The oligonucleotide linker 18N includes the nucleotide sequence of SEQ ID NO: 5 and the oligonucleotide linker 18NR includes the nucleotide sequence of SEQ ID NO: 6.

FIGS. 2A-2B include diagrams showing characterizations of exemplary HTA101-18N antibody-oligonucleotide conjugates. FIG. 2A: a photo showing results from reducing SDS-PAGE analysis of purified HTA101-18N with various oligonucleotide-to-antibody ratios (OARs) as indicated. M:molecular weight marker; U:unmodified IgG; OAR:oligo-to-antibody ratio; H:heavy chain; L:light chain; each lane contained 5.5 μg of antibody (excluding the weight of the oligonucleotides); gel was developed with InstantBlue Coomassie staining. FIG. 2B:photos showing mobility-shift assay of HTA101-18N ADCs. 18NR-HEX:complementary sequence conjugated to hexachlorofluorescein; 15NR-HEX:non-complementary control sequence. Lane 1:HTA101 IgG alone; Lane 2:HTA101+15NR-REX; Lane 3:HTA101+18NR-HEX; Lane 4-10:HTA101-18N (OAR 1.5, 1.9, 2.5, 2.9, 3.7, 4.6, 6.4)+18NR-HEX; Lane 11:HTA101-18N (OAR 6.4)+15NR-HEX. Each lane contained 40 nmole of IgG and 256 nmole (6.4 equivalences) of either oligonucleotide; 2% agarose gel, 0.5× TBE buffer; protein contents were visualized with Instant Blue Coomassie staining.

FIG. 3 includes photos showing internalization of HTA101-18N paired with 18NR-REX into SK-BR-3 cells visualized by confocal microscopy. Cells treated with paired HTA101-18N/18NR-HEX (35 nM IgG) were fixed with 3.7% formaldehyde before staining. Red:hexachlorofluorescein (HEX); green:lysosomal associated membrane protein 2 (LAMP2) stained with CD107b Alexa Fluor488-conjugated antibody; white:actin stained with AlexaFluor633-conjugated phalloidin; blue: nuclei stained with DAPI.

FIGS. 4A-4D. includes graphs illustrating the dose response curve of the potency of modular AOC/drug system of this disclosure measured by WST-1 cell viability assay. Dose-response curves were fitted with the standard 4-parameter logistic model. SK-BR-3 and N87:HER 2-overexpressing cell lines; HEK293T: negative control cell line. For each antibody/drug combination, an equimolar mixture of AOC and ssDNA-drug was prepared according to their respective OARs. FIG. 4A:Chemical structures of all ssDNA-drug conjugates used as payloads. FIG. 4B:Control experiments showing HER2-targeting activities of our AOC/ssDNA-drug complexes were dependent on strand hybridization. Unmodified+18NR-vcMMAE: a physical mixture of unmodified HTA101 antibody and 18NR-vcMMAE; blocker:ten-fold excess of 18NR complementary strand with no toxic payloads. FIG. 4C:Effect of different OARs on the potencies of our AOC/drug combination.

FIG. 4D: Comparison of our AOC/drug combination to the marketed ADC Kadcyla.

FIG. 5 is a table summarizing EC₅₀ values obtained from the WST-1 cell viability assay. The difference in the units used for the AOC/drug combinations (upper half) and ssDNA-drug controls (lower half).

FIG. 6 include graphs showing structures of various drug compounds conjugated to the 18 NR strand.

FIG. 7 includes diagrams showing ELISA analysis of HTA101-18N antibody-oligonucleotide conjugates binding affinities. Antigen:HER2 extracellular domain (0.3 μg/well); blocking agent: 5% defatted milk; plate:Nunc Maxisorp 96-well plate. Signals were produced by horseradish peroxidase (HRP)-conjugated anti-human Fc antibody using 3,3′,5,5′-tetramethylbenzidine (TMB) as the substrate. Binding curves were fitted with the standard 4-parameter logistic model. Left panel. The EC₅₀ values are shown in the right panel.

FIGS. 8A-8B include diagrams showing HPLC and MALDI-TOF mass spectrometric analysis of all oligonucleotides used in this study. FIG. 8A:purity check of oligonucleotides purified by reverse-phase chromatography (Atlantis T3 5 μm 4.6×250 mm C18 column) (left panel) and elution condition (right panel). FIG. 8B:MALDI-TOF mass spectrums of purified oligonucleotide-conjugated drug compounds as indicated.

FIGS. 9A-9B include diagrams showing results from the Lactate dehydrogenase (LDH) cell death assay of SK-BR-3 cells treated with AOC/drug combinations of this disclosure and the marketed ADC Kadcyla. FIG. 9A:Dose-response curves of various AOC/drug combinations. Cell deaths were reported colorimetrically as percentages relative to positive LDH enzyme controls. Curves were fitted with the standard 4-parameter logistic model. FIG. 9B:Summary of EC₅₀ values obtained from LDH assay.

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

The present disclosure is based, at least in part, on the development of a strand-hybridization-based linker format for conjugating biomolecules and agents of interest to form biomolecule-drug conjugates.

Having phosphate backbones, oligonucleotides are highly-charged hydrophilic molecules and can potentially mitigate issues stemming from hydrophobic payloads, which is a common problem associated with development of drug conjugates e.g. antibody-drug-conjugates (ADCs). In addition, oligonucleotides are generally non-immunogenic, and can only enter cells through receptor-mediated endocytosis, which is known to be a highly inefficient process. As such, the oligonucleotide conjugates disclosed herein are expected to have minimized off target toxicities, which is another common problem associated with conventional ADCs.^{17,19-22} Furthermore, hybridization between complementary strands is a very rapid process, with rate constants estimated to be 10⁶M·^{1s-1} for typical primer-length oligonucleotides.^{23,24} In comparison, most biorthogonal “click chemistry” reactions frequently used for conjugations, such as Staudinger ligation or Copper-catalyzed azide-alkyne cycloaddition (CuAAC), have rate constants lying in the range of 10⁻⁴ to 10²M^{-1s-1 25}

Thus, the oligonucleotide conjugates provided herein are expected to confer at least the following potential benefits:high hydrophilicity, low immunogenicity, modularized drug attachment, rapid preparation, or a combination thereof.

As demonstrated in the working examples, exemplary antibody-oligonucleotide complexes (or called antibody-oligonucleotide conjugates, AOCs) successfully paired with therapeutic agents conjugated with complementary strands rapidly and in a sequence-specific manner as observed by mobility-shift assays on agarose gel. Indirect ELISA showed that the antibody moiety in the exemplary ADC retained its binding ability to its target antigen. Further, confocal microscopy confirmed that the therapeutic agent carried by the ADC was successfully internalized into cancer cells. The in vitro cytotoxicity assays disclosed herein showed that the exemplary AOC disclosed herein can be used as a modular platform for drug delivery through hybridization a complementary strand conjugated with a variety of cargos.

This indicates that the oligonucleotide conjugate system disclosed herein would be a flexible drug-delivery strategy and platform for, e.g., therapeutic or diagnostic purposes.

1. Oligonucleotide Conjugates

In one aspect, the present invention discloses a first oligonucleotide conjugate (biomolecule-oligonucleotide conjugate) which comprises a first single strand oligonucleotide conjugate to a biomolecule wherein the first single strand oligonucleotide comprises a first nucleotide sequence. The present invention also discloses a second oligonucleotide conjugate (agent-oligonucleotide conjugate) comprising a second single strand oligonucleotide conjugated to an agent, wherein the second single strand oligonucleotide comprises a second nucleotide sequence being complementary to the first nucleotide sequence. The present invention further provides a double-strand oligonucleotide conjugate (biomolecule-oligonucleotides-agent) which comprises a hybridized oligonucleotide bridge region between the first nucleotide sequence and the second nucleotide sequence, whereby the biomolecule and the agent are linked together in the double-strand oligonucleotide conjugate.

As described herein, the term “polynucleotide” or “nucleic acid” refers to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. Polynucleotides or nucleic acids can be either single-stranded (e.g. ssRNA or a single-stranded cDNA) or double-stranded (e.g. a RNA/DNA duplex or dsDNA). It will be understood that when a nucleotide sequence is represented by a DNA sequence (i.e., A, T, G, C), this also includes an RNA sequence (i.e., A, U, G, C) in which “U” replaces “T.” The term “oligonucleotide” refers to a relatively short nucleic acid fragment, typically less than or equal to 150 nucleotides long. Oligonucleotides can be designed and synthesized as needed.

As described herein, the term “complementary” with respect to nucleotide sequences include the meanings of the topological compatibility or matching together of interacting surfaces of two polynucleotides. Thus, the two molecules can be described as complementary, and furthermore the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. Thus, the polynucleotide whose sequence 5′-TATAC-3′ is complementary to a polynucleotide whose sequence is 5′-GTATA-3′.

As used herein, the term “substantially identical” refers to two sequences having 70% or more, preferably 75% or more, more preferably 80% or more, even more preferably 85% or more, still even more preferably 90% or more, and most preferably 95% or more or 100% identity.

To determine the percent identity of two sequences, the sequences are aligned for optimal comparison purposes (e.g., gaps can be introduced in the sequence of a first nucleotide sequence for optimal alignment with a second nucleotide sequence). In calculating percent identity, typically exact matches are counted. The determination of percent homology or identity between two sequences can be accomplished using a mathematical algorithm known in the art, such as BLAST and Gapped BLAST programs, the NBLAST and)(BLAST programs, or the ALIGN program.

As used herein, the term “melting temperature (Tm)” refers to a temperature at which one-half of a nucleic acid duplex dissociates generating single strand polynucleotide.

As described herein, the term “hybridization” as used herein shall include any process by which a strand of nucleic acid joins with a complementary strand through base pairing. Relevant methods are well known in the art and described in, for example, Sambrook et al., Molecular Cloning: A Laboratory Manual, 2nd ed., Cold Spring Harbor Laboratory Press (1989), and Frederick M. A. et al., Current Protocols in Molecular Biology, John Wiley & Sons, Inc. (2001). Typically, stringent conditions are selected to be about 5 to 30° C. lower than the thermal melting point (Tm) for the specified sequence at a defined ionic strength and pH. More typically, stringent conditions are selected to be about 5 to 15° C. lower than the T m for the specified sequence at a defined ionic strength and pH. For example, stringent hybridization conditions will be those in which the salt concentration is less than about 1.0 M sodium (or other salts) ion, typically about 0.01 to about 1 M sodium ion concentration at about pH 7.0 to about pH 8.3 and the temperature is at least about 25° C. for short oligonucleotides (e.g., 10 to 50 nucleotides) and at least about 55° C. for long oligonucleotides (e.g., greater than 50 nucleotides). An exemplary non-stringent or low stringency condition for a long oligonucleotides (e.g., greater than 50 nucleotides) would comprise a buffer of 20 mM Tris, pH 8.5, 50 mM KCl, and 2 mM MgCl₂, and a reaction temperature of 25° C.

In some embodiments, the one or both oligonucleotides described herein may contain non-naturally-occurring nucleobases, sugars, or covalent internucleoside linkages (backbones). Such a modified oligonucleotide confers desirable properties such as enhanced cellular uptake, improved affinity to the target nucleic acid, and increased in vivo stability.

In one example, the oligonucleotides described herein has a modified backbone, including those that retain a phosphorus atom (see, e.g., U.S. Pat. Nos. 3,687,808; 4,469,863; 5,321,131; 5,399,676; and 5,625,050) and those that do not have a phosphorus atom (see, e.g., U.S. Pat. Nos. 5,034,506; 5,166,315; and 5,792,608). Examples of phosphorus-containing modified backbones include, but are not limited to, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having 3′-5′ linkages, or 2′-5′ linkages. Such backbones also include those having inverted polarity, i.e., 3′ to 3′, 5′ to 5′ or 2′ to 2′ linkage. Modified backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In some instances, the modified backbone can be an N-2-aminoethylglycine backbone (peptide nucleic acid or PNA).

In another example, the oligonucleotides described herein include one or more substituted sugar moieties. Such substituted sugar moieties can include one of the following groups at their 2′ position:OH; F; O-alkyl, S-alkyl, N-alkyl, O-alkenyl, S-alkenyl, N-alkenyl; O-alkynyl, S-alkynyl, N-alkynyl, and O-alkyl-O-alkyl. In these groups, the alkyl, alkenyl and alkynyl can be substituted or unsubstituted C1 to C10 alkyl or C2 to C10 alkenyl and alkynyl. They may also include at their 2′ position heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide. Preferred substituted sugar moieties include those having 2′-methoxyethoxy, 2′-dimethylaminooxyethoxy, and 2′-dimethylaminoethoxyethoxy. See Martin et al., Helv. Chim. Acta, 1995, 78, 486-504.

Alternatively or in addition, the oligonucleotides described herein may include one or more modified native nucleobases (i.e., adenine, guanine, thymine, cytosine and uracil). Modified nucleobases include those described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and Sanghvi, Y. S., Chapter 15, Antisense Research and Applications, pages 289-302, CRC Press, 1993.

In some embodiments, the first and second nucleotide sequences each comprises a GC rich sequence. In some embodiments, the first nucleotide sequence and the second nucleotide sequence each has substantially no secondary structure.

As described herein, the term “GC-rich” as used herein refer to a polynucleotide or an oligonucleotide having a relatively high number of G and/or C bases in its structure, or in a part or region of its structure. In general, oligonucleotides having nucleotide sequences greater than about 35% GC content are considered GC-rich sequences. For example, GC-rich sequences are those presenting GC content of 35% to 75%, such as 40% to 75%, 45% to 75%, 50% to 75%, 55% to 75%, 60% to 75% or 65% to 75%.

As described herein, the phrase “having substantially no secondary structure” with respect to a single strand oligonucleotide include the meaning that the oligonucleotide does not have a sequence whereby there are substantial portions being inverse complementary to each other (i.e. one region of the oligonucleotide to hybridize with another region) and thus allowing for intramolecular base pairing. The term “substantial portions” may include the meaning of four (4) or more consecutive nucleotide residues. In particular, a single strand oligonucleotide as used herein for carrying a biomolecule or an agent of interest is designed to avoid such secondary structure e.g. a loop. Preferably, a single strand oligonucleotide as used herein only performs complement of its binding partner and does not include the sequences to generate secondary structures itself

The oligonucleotides disclosed herein may have a suitable length. In some embodiments, one or both of the oligonucleotides may contain about 12 to about 80 nucleotides (nts), for example, about 15 to about 60 nts, about 15 to about 50 nts, about 15 to about 40 nts, about 15 to about 30 nts, about 15 nts to about 25 nts, or about 15 to about 20 nts. In some examples, the one or both oligonucleotides may contain about 15 to about 25 nts, for example, about 18 nts.

The two oligonucleotides in the hybridized oligonucleotide conjugate disclosed herein may be of the same length, or may have different lengths. In some examples, the whole sequence of one oligonucleotide is complementary to the whole or part of the other oligonucleotide. In other examples, a portion of one oligonucleotide is complementary to the whole or part of the other oligonucleotide.

In some instances, the two oligonucleotides comprise completely complementary sequences (i.e., with no mismatched base pairs). Alternatively, the two oligonucleotides may comprise partially complementary sequences (i.e., comprising one or more mismatched base pairs) while still capable of forming a double-stranded structure. The level of tolerable mismatching that would not affect formation of a double-stranded structure in a particular sequence is known to those skilled in the art.

In some embodiments, the first and second nucleotide sequences individually comprises a GC rich sequence, ranging from 12 nucleotides (nts) to 80 nts in length (such as 15 to 60 nts, 15 to 50 nts, 15 to 40 nts, 15 to 30 nts, 15 to 25 nts, 15 to 20 nts, or 18 nts) and having substantially no secondary structure. Particularly, the first and second nucleotide sequences have a melting temperature (Tm) of at least 38° C., such as 40° C.−70° C. (e.g. 41° C.-69° C., 43° C.−67° C., 45° C.−65° C., 47° C.−63° C., 49° C.−60° C., 51° C.−59° C. or 53° C.−57° C.).

In some particular embodiments, the first and second nucleotide sequences individually comprises a nucleotide sequence which comprises a motif of 5′-SSWSSWSWSSSWWSSWSS-3′ (SEQ ID NO: 1) wherein each S is independently selected from G or C and each W is independently selected from A or T. Examples of such sequence include 5′-GGWCCWGWCCGWWGGWCC-3′ (SEQ ID NO: 3) such as 5′-GGACCAGACCGAAGGACC-3′ (SEQ ID NO: 5). The first and second nucleotide sequences as described herein may also include a substantially identical sequence to the particular sequence no. as described herein e.g. a nucleotide sequence having 70% or more, preferably 75% or more, more preferably 80% or more, even more preferably 85% or more, still even more preferably 90% or more, and most preferably 95% or more or 100% identity to SEQ ID NO: 1, 3 or 5.

In some particular embodiments, the first and second nucleotide sequences individually comprises a GC rich sequence which comprises a motif of 5′-SSWSSWWSSSWSWSSWSS-3′ (SEQ ID NO: 2) wherein each S is independently selected from G or C and each W is independently selected from A or T. Examples of such GC rich sequence include 5′-GGWCCWWCGGWCWGGWCC-3′ (SEQ ID NO: 4) such as 5′-GGTCCTTCGGTCTGGTCC-3′ (SEQ ID NO: 6). The first and second nucleotide sequences as described herein may also include a substantially identical sequence to the particular sequence no. as described herein e.g. a nucleotide sequence having 70% or more, preferably 75% or more, more preferably 80% or more, even more preferably 85% or more, still even more preferably 90% or more, and most preferably 95% or more or 100% identity to SEQ ID NO: 1, 3 or 5.

As described herein, a biomolecule with respect to conjugation to an oligonucleotide to generate a biomolecule-oligonucleotide conjugate is preferably a targeting molecule which functions in recognizing a particular target (for example, a disease-associated antigen such as a tumor antigen) so as to localize at a target area, enter a target cell and/or bind to a target antigen or receptor. In some instances, the targeting biomolecules can be antibodies, nucleic acids (e.g., aptamers), lectins, adhesion molecules, cytokines, saccharides, steroids, hormones, peptides, proteins, and enzymes.

As described herein, an agent of interest with respect to conjugation to an oligonucleotide to generate an agent-oligonucleotide conjugate can be a molecule of any type having a desired utility e.g. therapeutic utility, diagnostic utility or cosmetic uses. In some instances, the agent of interest is a therapeutic agent, which can be any molecule having therapeutic effects against a target disease or disorder. In some examples, the therapeutic agent may be a small molecule cytotoxic agent such as anti-cancer drugs e.g. monomethyl auristatin E (MMAE) or mertansine (DM1). In some examples, an agent of interest may be a peptide-based or polypeptide-based molecule such as an antibody or a targeting peptide. For example, anti-cancer antibodies include but are not limited to anti-HER2 antibodies, anti-VEGF antibodies, anti-CD20 antibodies, anti-ErbB2 antibodies and anti-CD30 antibodies. In some examples, an agent of interest may be a diagnostic agent, which can be any moiety possessing a property or function which can be used for detection purposes, such as a fluorescent moiety (e.g. polyfluorenes, fluorescein, or Ru, Eu, Pt complexes), a luminescent moiety (e.g. a horseradish peroxidase label) or a radioactive moiety (e.g. tritium (³H), ³²P, ³⁵S or ¹⁴C, or covalently bound labels, such as ¹²⁵I bound to tyrosine, ¹⁸F within fluorodeoxyglucose, or metallo-organic complexes e.g. ⁹⁹Tc-DTPA).

In some embodiments, the first single strand oligonucleotide is conjugated at 3′-end to the targeting biomolecule, and/or the second single strand oligonucleotide is conjugated at 3′-end to the agent.

In some embodiments, the first single strand oligonucleotide is conjugated at 5′-end to the targeting biomolecule, and/or the second single strand oligonucleotide is conjugated at 5′-end to the agent.

According to the present invention, an oligonucleotide conjugate as described herein can be provided with various molar ratio between the biomolecule/agent and the oligonucleotide in the conjugate, which can be measured by methods known in the art e.g. SDS-PAGE. In some instances, the molar ratio between the biomolecule and the first oligonucleotide in the oligonucleotide conjugate may range from 1:1 to 1:6 (e.g. 1:1, 1:2, 1:3, 1:4, 1:5 or 1:6).

The oligonucleotide conjugate system disclosed herein performs as a flexible drug-delivery strategy and platform to prepare an oligonucleotide conjugate of desired functions by choosing a suitable biomolecule and an agent of interest to be conjugated with the oligonucleotides as needed. For example, a biomolecule and/or an agent of interest to be conjugated is an antibody, and when both are antibodies, they may target different antigens, thus providing a bispecific antibody molecule. According to the present invention, the oligonucleotide linkage approach as described herein to create dual or multifunctional molecule is not limited to antibodies but can be utilized to connect any two molecules such as adhesion molecules, cytokines or lectins

In particular embodiments, the present invention provides an antibody-oligonucleotide conjugate (AOC) when a targeting molecule to be conjugated to an oligonucleotide is an antibody. In some instance, an AOC is hybridized with an agent-oligonucleotide conjugate, forming a double strand antibody-drug conjugate (ADC), each strand comprising an antibody and an agent of interest, respectively, whereby the antibody and agent of interest are conjugated via a double-stranded oligonucleotide-based linker.

As used herein, an antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (e.g., full-length) polyclonal or monoclonal antibodies, but also antigen-binding fragments thereof (such as Fab, Fab′, F(ab′)₂, Fv), single-chain antibody (scFv), fusion proteins comprising an antibody portion, humanized antibodies, chimeric antibodies, diabodies, single domain antibody (e.g., nanobody), single domain antibodies (e.g., a V_{H} only antibody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinforg.uk/abs).

The antibody in any of the ADCs disclosed herein may be a full-length antibody, which contains two heavy chains and two light chains, each including a variable domain and a constant domain. Alternatively, the antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, CL and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The antibodies described herein can be of a suitable origin, for example, murine, rat, or human. Such antibodies are non-naturally occurring, i.e., would not be produced in an animal without human act (e.g., immunizing such an animal with a desired antigen or fragment thereof or isolated from antibody libraries). Any of the antibodies described herein, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the antibodies are human antibodies, which may be isolated from a human antibody library or generated in transgenic mice. In other embodiments, the antibodies may be humanized antibodies or chimeric antibodies.

2. Preparation of Oligonucleotide Conjugates

The oligonucleotide conjugates as described herein can be prepared via routine procedures, e.g., recombinant technology, hybridoma technology, chemical synthesis, etc.

To prepare any of the oligonucleotide conjugates disclosed herein, an oligonucleotide can be conjugated onto a biomolecule via routine practice or methods provided herein to produce a biomolecule-oligonucleotide conjugate and a complementary oligonucleotide can be conjugated to an agent of interest following knowledge known in the art or guidance provided herein. The two oligonucleotide conjugates can then be incubated together under conditions allowing for hybridization of the two oligonucleotides to produce a hybrid, double-strand oligonucleotide conjugate where the biomolecule and the agent are linked together.

As described herein, conjugation of oligonucleotides to biomolecules or agents of interest may be performed covalently or non-covalently. Methods for covalently or non-covalently conjugation are available in this art. Non-covalent linkage may be performed by ionic interactions such as a protamine charge-force approach and affinity binding such as an avidin-based conjugation approach. In case of a covalent linkage between a biomolecule/agent moiety and an oligonucleotide, a direct reaction of an activated group either on the biomolecule/agent moiety or on oligonucleotide with an functional group on either the oligonucleotide or on the biomolecule/agent moiety or via an heterobifunctional linker molecule, which is firstly reacted with one and then reacted with the other binding partner, is possible. Examples of the chemical linker include but are not limited to a succinimide moiety, a maleimide moiety, a hydrazine moiety, a tyrosine moiety, a hydrazone moiety, an azide moiety, a terminal alkyne moiety, a strained terminal alkyne moiety, or a phosphine moiety. The conjugation may occur at the 5′-end or 3-end of the oligonucleotides to biomolecules or agents of interest.

In some embodiment, an oligonucleotide for use in making the oligonucleotide conjugate disclosed herein may be modified to add a functional handle, which can react with the biomolecule, the agent of interest, or a linker (e.g., a chemical linker) to form a covalent bond. A functional handle can be any chemical moiety comprising a functional group that can react with another functional group to form covalent bonds. Exemplary functional groups include, but are not limited to, a hydroxyl group (—OH), a methyl group, a carbonyl group (—C═O), a carboxyl group (—COOH), an amino group (—N), a phosphate group, or a thiol group (—SH). The functional handle may be added to the 5′ end of the oligonucleotides. Alternatively, it may be added to the 3′ end of the oligonucleotides.

In some examples, an oligonucleotide carrying a functional handle may be linked directly to a functional group carried by an amino acid residue in the antibody. Alternatively, the oligonucleotide carrying the functional handle may be linked to a functional group carried by an amino acid residue in the antibody via a chemical linker. Examples of functional groups in the antibody include the —OH group in tyrosine or serine, the —NH₂ group in lysine, arginine, or histidine, the —COOH group in aspartic acid or glutamic acid, or the —SH group in cysteine. In some examples, the antibody may comprise one or more internal disulfide bonds. Such an antibody may be treated by a reducing agent to release the —SH functional group for conjugation with the oligonucleotide, either directly or via a chemical linker. Exemplary chemical linkers may comprise, without limitation, a succinimide moiety, a maleimide moiety, a hydrazine moiety, a tyrosine moiety, or a hydrazone moiety.

Other approaches for conjugating an oligonucleotide onto an antibody are known in the art and can be used in the disclosures provided herein. See, e.g., WO2017/190020, the relevant disclosures of which are incorporated by reference for the purpose and subject matter referenced herein.

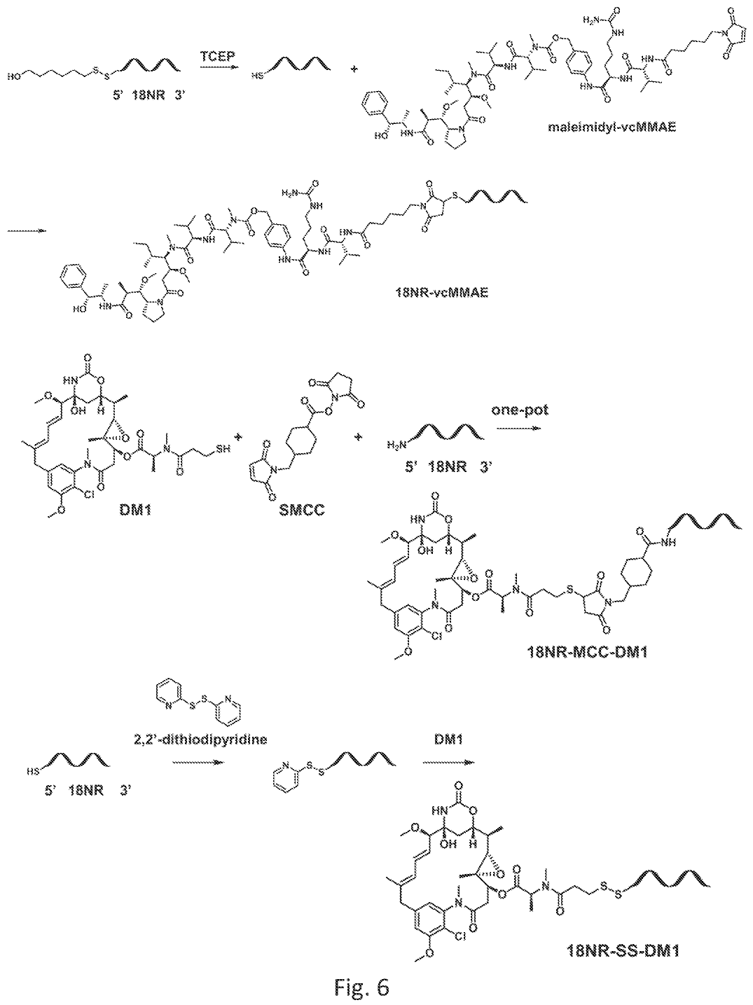

Conjugating an oligonucleotide to an agent of interest would depend on the nature of the agent of interest, e.g., chemical structures thereof. In some examples, the agent of interest comprises a functional group that is reactive to the functional handle carried by an oligonucleotide as disclosed herein. In that case, a direct reaction between the agent of interest and the oligonucleotide carrying the functional handle can be taken place to conjugate the agent of interest with the oligonucleotide. In other cases, the agent of interest may be modified to add a second functional handle that is reactive to the functional handle linked to the oligonucleotide. Examples are provided in FIG. 6, using MMAE and DM1 as exemplary cytotoxic agents.

The biomolecule-oligonucleotide conjugate and the oligonucleotide-conjugated agent of interest can then be incubated under suitable hybridization conditions to allow for formation of a double-stranded structure between the complementary oligonucleotides, thereby forming an oligonucleotide conjugate in a hybrid form linked with the biomolecule and the agent of interest as disclosed herein. The suitable hybridization conditions (e.g., temperature, ion strength, incubation time etc.) would be determined based on various factors, for example, length of the oligonucleotides, melting temperature of the oligonucleotides, level of complementarity, etc., which are within the knowledge of those skilled in the art.

Exemplary procedures for making the oligonucleotide conjugate disclosed are provided in the Example 1.

3. Uses of Oligonucleotide Conjugate

Any of the oligonucleotide conjugate as described herein can be mixed with a pharmaceutically acceptable carrier to form a pharmaceutical composition for use, e.g., in treating or diagnosing a target disease or detecting a target (e.g. a disease-associated antigen). “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. See, e.g., Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The pharmaceutical compositions to be used for in vivo administration must be sterile. This is readily accomplished by, for example, filtration through sterile filtration membranes. Compositions comprising the conjugate may be placed into a container having a sterile access port, for example, an intravenous solution bag or vial having a stopper pierceable by a hypodermic injection needle.

Any of the conjugate described herein can be used to deliver the agent of interest contained therein to specific cells and/or tissues to which the antibody component targets. To practice the method disclosed herein, an effective amount of the pharmaceutical composition described herein that contains any of the oligonucleotide conjugates as also disclosed herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. As used herein, “an effective amount” refers to the amount of each active agent required to confer therapeutic effect on the subject, either alone or in combination with one or more other active agents. Determination of whether an amount of the oligonucleotide conjugate disclosed herein achieved the therapeutic or diagnostic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder. Alternatively, sustained continuous release formulations of a conjugate as disclosed herein may be appropriate. Various formulations and devices for achieving sustained release are known in the art.

For the purpose of the present disclosure, the appropriate dosage of an oligonucleotide conjugate as described herein will depend on the specific therapeutic or diagnostic agent contained in the oligonucleotide conjugate, the biomolecule component in the oligonucleotide conjugate, the type and severity of the disease/disorder, whether the oligonucleotide conjugate is administered for preventive or therapeutic purposes, previous therapy, the patient's clinical history and response to the antagonist, and the discretion of the attending physician. A clinician may administer an oligonucleotide conjugate until a dosage is reached that achieves the desired result. In some embodiments, the desired result is improvement of at least one symptom associated with a target disease or disorder or diagnosis of at least one biomarker associated with a target disease/disorder. Methods of determining whether a dosage resulted in the desired result would be evident to one of skill in the art. Administration of one or more oligonucleotide conjugate doses can be continuous or intermittent, depending, for example, upon the recipient's physiological condition, whether the purpose of the administration is therapeutic or prophylactic, and other factors known to skilled practitioners. The administration of an oligonucleotide conjugate may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

As used herein, the term “diagnosis” as used herein generally includes determination as to whether a subject is likely affected by a given disease, disorder or dysfunction. The skilled artisan often makes a diagnosis on the basis of one or more diagnostic indicators, i.e., a marker, the presence, absence, or amount of which is indicative of the presence or absence of the disease, disorder or dysfunction.

Conventional methods, known to those of ordinary skill in the art of medicine, can be used to administer the pharmaceutical composition to the subject, depending upon the type of disease to be treated or diagnosed, or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularlly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, a water soluble conjugate can be administered by the drip method, whereby a pharmaceutical formulation containing the conjugate and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the oligonucleotide conjugate, can be dissolved and administered in a pharmaceutical excipient such as Water-for-Injection, 0.9% saline, or 5% glucose solution.

In one embodiment, a conjugate is administered via site-specific or targeted local delivery techniques. Examples of site-specific or targeted local delivery techniques include various implantable depot sources of the conjugate or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No.

The subject to be treated by the methods described herein can be a mammal, such as a farm animals, sport animals, pets, primates, horses, dogs, cats, mice and rats. In one example, the subject is a human. The conjugate-containing composition may be used for treating or diagnosing a target disease or disorder. In some examples, the subject may be a human patient having, suspected of having, or at risk for a target disease or disorder, for example, cancer. Such a patient can also be identified by routine medical practices.

A subject having a target disease or disorder (e.g., cancer) can be identified by routine medical examination, e.g., laboratory tests, organ functional tests, CT scans, or ultrasounds. A subject suspected of having any of such target disease/disorder might show one or more symptoms of the disease/disorder. A subject at risk for the disease/disorder can be a subject having one or more of the risk factors associated with that disease/disorder. Such a subject can also be identified by routine medical practices.

The particular dosage regimen, i.e., dose, timing and repetition, used in the method described herein will depend on the particular subject (e.g., a human patient) and that subject's medical history.

In some embodiments, the conjugate disclosed herein may be co-used with another suitable therapeutic agent for the target disease or disorder. Alternatively or in addition, the conjugate disclosed herein may also be used in conjunction with other agents that serve to enhance and/or complement the effectiveness of the agents.

4. Kit for Drug Delivery

The present disclosure also provides kits for use in delivering an agent of interest to a subject in need of the treatment using any of the oligonucleotide conjugates disclosed herein that comprise the agent of interest. Such kits can include one or more containers comprising an oligonucleotide conjugate, e.g., any of those described herein.

In some embodiments, the kit can comprise instructions for use in accordance with any of the methods described herein. The included instructions can comprise a description of administration of the conjugate to treat, delay the onset, or alleviate a target disease as those described herein. The kit may further comprise a description of selecting an individual suitable for treatment based on identifying whether that individual has the target disease. In still other embodiments, the instructions comprise a description of administering the conjugate to an individual at risk of the target disease.

The instructions relating to the use of an oligonucleotide conjugate generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a disease or disorder associated with cancer, such as those described herein. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an oligonucleotide conjugate as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

5. General techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds.(1985»; Transcription and Translation (B. D. Hames & S. J. Higgins, eds. (1984»; Animal Cell Culture (R. I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

Development of A Flexible and Modular Linker Strategy for Antibody-drug Conjugates Based on Oligonucleotide Strand-Pairing

Linker design is crucial to the success of antibody-drug conjugates (ADCs). This example provides an exemplary modular linker format for attaching molecular cargos to antibody based on strand-pairing between complementary oligonucleotides. Briefly, antibody-oligonucleotide conjugates (ADCs) were prepared by attaching an 18-mer oligonucleotide to an anti-HER2 antibody as an example through the thiol-maleimide chemistry, which is an approach generally applicable to any immunoglobulins with internal disulfide bridges. The AOC thus produced was then hybridized to a drug-bearing oligonucleotide that is complementary to the oligonucleotide moiety in the AOC, thereby producing the ADC. This hybridization process was rapid, stoichiometric, and sequence-specific.

In this work, we present the preparation and characterization of AOCs consisting of an anti-HER2 IgG1 antibody (HTA101, derived from murine antibodies repertoires and subsequently humanized) and covalently-bonded GC-rich 18-mer ssDNA strands (18N).²⁶ The sequence of 18N was designed to have a melting temperature above 55 degree Celsius and no predictable secondary structures. Our AOCs were hybridized to their complementary sequence bearing various cytotoxic drugs, including monomethyl auristatin E (MMAE) and mertansine (DM1), and evaluated for in vitro potencies against HER2-overexpressing cancer cell lines.

The results provided herein indicate that the ADCs thus produced were able to selectively target HER2-overexpressing cell lines such as SK-BR-3 and N87, with in vitro potencies similar to that of the marketed ADC Kadcyla (T-DM1).

This study demonstrated the potential of utilizing AOCs as a highly flexible and modular platform, where a panel of well-characterized AOCs bearing DNA, RNA, or various nucleic acid analogs such as peptide nucleic acids could be paired with any cargo of choice at ease for a wide range of diagnostic or therapeutic applications.

Abbreviations:ACN, acetonitrile; ADC, antibody-drug conjugate; AOC, antibody-oligonucleotide conjugate, CuAAC, copper-catalyzed azide-alkyne cycloaddition, DAR, drug-to-antibody ratio; DIPEA, diisopropylethylamine; DM1, mertansine; DMSO, dimethyl sulfoxide; HEPES, 4-(2-hydroxyethyl)-1-piperazineethanesulfonic acid; HEX, hexachlorofluorescein; LAMP2, lysosome-associated membrane protein 2; LDH, lactate dehydrogenase; OAR, oligonucleotide-to-antibody ratio; PBS, phosphate buffered saline; SMCC, succinimidyl 4-(N-maleimidomethyl)cyclohexane-1-carboxylate; TCEP, tris(2-carboxyethyl)phosphine; TEA:trimethylamine; TEAA, triethylammonium acetate; Tris, tris(hydroxymethyl)aminomethane; vcMMAE:valine-citrulline monomethyl auristatin E

1. Materials and Methods

1.1 General Material and Instrumentation.

Oligonucleotides were ordered from Integrated DNA technologies (IDT). Cytotoxic drugs, including vcMMAE and DM1, were ordered from Medchem Express and Medcoo Biosciences. Most chemicals and solvents were purchased from Thermo Fisher Scientific and Sigma Aldrich. High performance liquid chromatography was performed on a Dionex Ultimate 3000 system. UV-vis spectroscopy was performed on a Nanodrop 1000 spectrometer. Mass spectrometry was performed on a Bruker Autoflex III MALDI-TOF/TOF mass spectrometer equipped with a 200 Hz SmartBean Laser in positive ion mode. Fluorescent images of agarose gels were captured on a BioDoc-It Imaging System. N87 and SK-BR-3 cell lines were purchased from American Type Culture Collection (ATCC). Calculation of EC₅₀ values and curve-fitting was performed with Prism data analysis software and Origin data analysis software.

### 1.2 Preparation of 18N-MCC

18N Oligonucleotide with a 5′ end amino modifier in ddH₂O (3.62 mM, 15.70 μL) was diluted with ddH₂O to 20 μL and mixed well with DIPEA (3.48 μL). SMCC cross-linker freshly dissolved in DMSO (100 mM, 10 μL) was subsequently added. The reaction was diluted with DMSO to a final oligonucleotide concentration of 500 μM, with a total volume of 100 μL. The reaction was stirred at room temperature for 2 hours. Upon completion, particulate matter was removed by centrifugation at 16000 g for 10 minutes, and the crude 18N-MCC oligonucleotide was further purified by reverse-phase HPLC (Atlantis T3 5 μm 4.6×250 mm C18 column) using the TEAA/ACN (0.1 M, pH 7.0) system. Fractions containing pure 18N-MCC were combined, concentrated, and lyophilized as a white solid for future use.

### 1.3 Preparation of 18NR-vcMMAE

18NR Oligonucleotide with a 5′ end disulfide modifier was mixed with sodium phosphate buffer (1 M, pH 7.2, 20 μL) and aqueous TCEP (100 mM, 20 μL). The reaction was diluted with deionized water to a final oligonucleotide concentration of 266 μM, with a total volume of 100 μL. The reduction was allowed to proceed at room temperature for 1 hour with stirring. Upon completion, sodium acetate (3 M, pH 5.2, 40 μL) and 95% ethanol (280 μL) were added, resulting in a cloudy precipitation of oligonucleotides. The precipitate was spun down at 16000 g for 10 minutes, washed once with 75% ethanol (200 μL), and reconstituted in deionized water (100 μL). The oligonucleotide was then mixed with a freshly prepared DMSO solution of vcMMAE (100 mM, 20 μL) and further diluted with DMSO (150 μL). The mixture was allowed to stand at room temperature for 1 hour. The crude oligonucleotide was further purified by reverse-phase HPLC, using the same method described above. The lyophilized solid was precipitated once more with sodium acetate to remove residual triethylammonium salt, dissolved in deionized water, and stored at 4 as a stock solution.

### 1.4 Preparation of 18NR-SS-DM1

SMCC cross-linker freshly dissolved in DMSO (100 mM, 20 μL) was combined and mixed well with a DMSO solution of DM1 (100 mM, 20 μL). The mixture was allowed to stand at room temperature for 30 minutes. To the mixture was added 18NR oligonucleotide in deionized water (3.72 mM, 53.76 μL) and sodium phosphate buffer (1 M, pH 7.2, 40 μL). The reaction was further diluted by deionized water (106.24 μL) and ACN (160 μL) and stirred at room temperature for 2 hours. Tris buffer (1M, pH 7.5, 40 μL) was added to quench the reaction. The reaction mixture was filtered, and the crude 18N-MCC-DM1 was further purified by reverse-phase HPLC. Removal of triethylammonium salt and subsequent storage followed that of 18NR-SS-DM1.

### 1.5 Preparation of IgG-18N and Determination of OAR

To a stock solution (450 μL) of HTA101 anti-HER2 antibody (2 mg/mL, 50 mM HEPES, 150 mM NaCl) was added EDTA solution (0.5M, pH 8.0, 9 μL) and TCEP solution (10 mM, 10 equivalences with respect to the number of inter-chain disulfide bonds). Reduction was allowed to proceed on a rotatory mixer and reacted at room temperature for 2 hours. 18N-MCC dissolved in ultrapure water was added to the reduced antibody at various equivalences (2 to 8), and the conjugation was performed on the rotatory mixer overnight. Unconjugated antibodies and other impurities were removed by anion exchange chromatography (Protein-pak Hi-res Q, 5 μm, 4.6×100 mm, 0 to 2 M NaCl gradient), followed by size-exclusion chromatography (Ultrahydrogel 250) to remove residual unconjugated oligonucleotides. Oligonucleotide-to-antibody ratios were determined by measuring UV-vis absorbances at 260 nm and 280 nm using the following equations and extinction coefficients:

C_{IgG}·E_{260 nm},I_{g}G+C_{18N}·E_{260 nm},18N=A₂₆₀

C_{IgG}·E_{280 nm},I_{g}G+C_{18N}·E_{280 nm},18N=A₂₈₀

E_{260 nm,IgG}=115972M⁻¹cm⁻¹;

E_{280 nm,IgG}=218404M⁻¹cm⁻¹

E_{260 nm},18N=181100M⁻¹cm⁻¹;

E_{280 nm},18N=99396M⁻¹cm⁻¹

where C_{IgG} and C18N are the individual molarities of the antibody and the oligonucleotide, respectively. The purified HTA101-18N with various OARs were filtered through 0.22 μm membrane and stored in phosphate-buffered saline (pH 7.2) at 4.

### 1.6 HTA101-18N Paired with Toxin-bearing Complementary Strands

The hybridization of drug-bearing complementary oligonucleotides to AOCs was incubated in PBS buffer (pH 7.4) at room temperature for 30 mins.

### 1.7 Cell Culture and Cytotoxicity Assays

N87 and SK-BR-3 cells were grown in RPMI 1640 and DMEM media (Invitrogen) supplemented with 10% fetal bovine serum at 37 in a humidified atmosphere containing 5% carbon dioxide. SK-BR-3 and N87 were seeded at densities of 10⁴ cells/well and 3×10⁴ cells/well, respectively. After cell attachment to the well, AOCs paired with toxin-bearing complementary strands were serially diluted and added. Cells were then incubated at 37° C. for 2 to 3 days. At the suitable time point, the cells were harvested for either LDH (Thermo Fisher Scientific) or WST-1 assay (Roche) following the manufacturers' instructions for viability determination. Viabilities or cell deaths were defined as percentages relative to untreated control cells or enzymes.

### 1.8 Imaging by Confocal Microscopy

SK-BR-3 cells were seeded on glass coverslips for 16 hours, which were then starved in serum-free medium for 2 hours. AOCs (OAR 4.7) paired with 18NR-HEX were added to the cells to a final concentration of 35 nM (in terms of antibody), followed by 12 hours of incubation. Cells were then fixed with 3.7% formaldehyde at room temperature for 15 minutes and stained with NucBlec Live ReadyProbes 405 (Thermo Fisher Scientific), CD107b-Akexa Fluor 488 (Thermo Fisher Scientific), and Alexa Fluor 633 Phalloidin (Thermo Fisher Scientific) following the manufacturer's protocols. Observation of fluorescence was performed on a Zeiss LSM 510 META confocal microscope equipped with a LD-Achroplan objective (20×/0.4 korrPh2×63×, 1.3xoi1).

### 2. Results

### 2.1 Preparation and Characterization of HTA101-18N Antibody-Oligonucleotide Conjugate

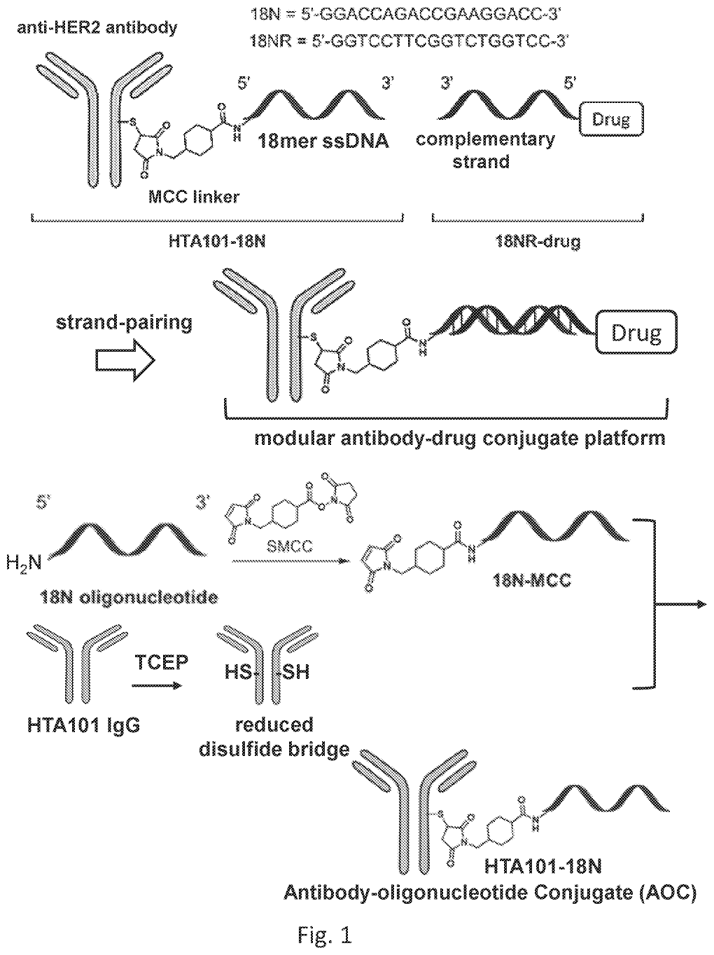

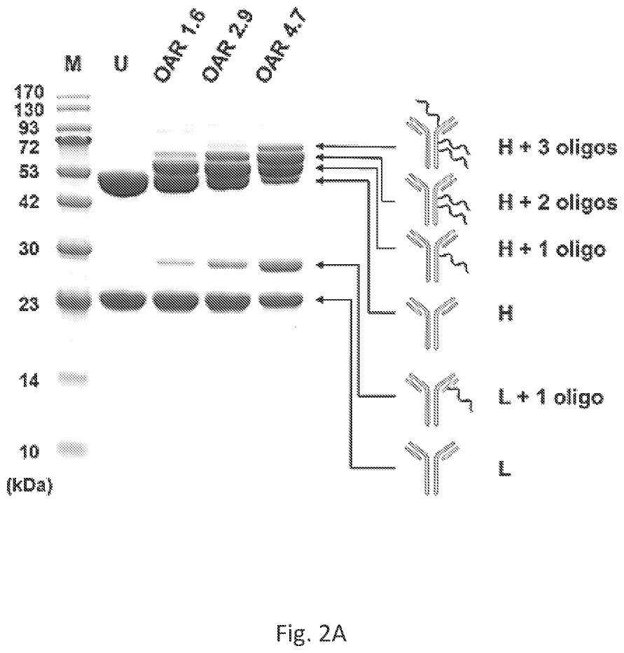

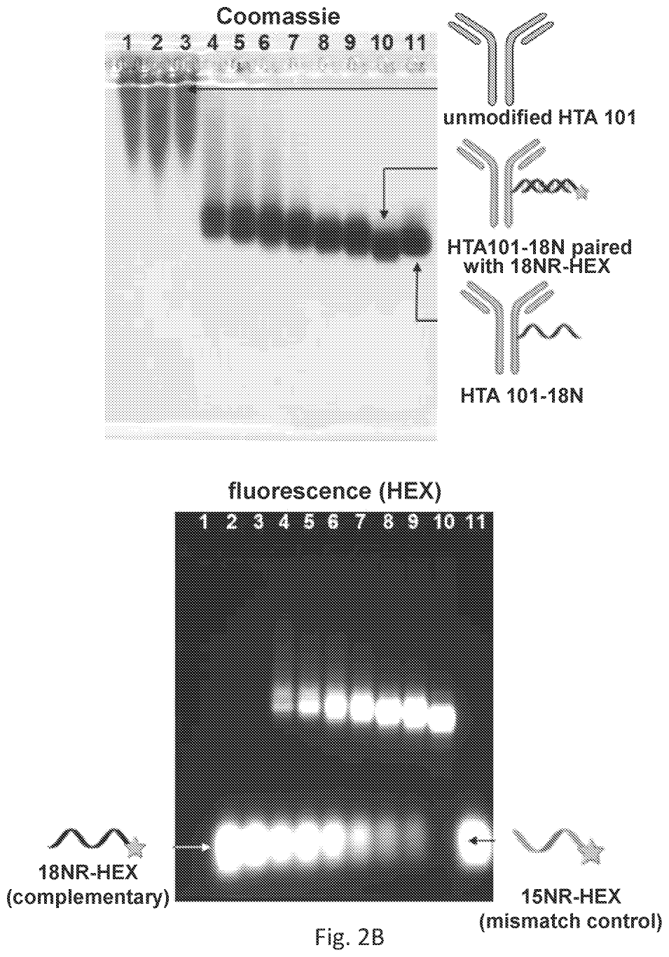

Two simplest ways to chemically modify antibodies without any genetically encoded tags are amide bond formation through surface lysine and Michael-addition through reduced inter-chain disulfide bonds. It has been recognized that conjugates prepared through sulfhydryl groups, due to their relative scarcity compared to lysine residues on an antibody, are far less heterogeneous, leading to more favorable properties.²⁷ The 5′ end primary amine of our 18N sequence was first functionalized with SMCC to generate a sulfhydryl-reactive maleimide handle. The resultant 18N-MCC was reacted with TCEP-treated HTA101 antibody at various molar equivalences to generate AOCs (HTA101-18N) with various oligonucleotide-to-antibody ratios (OARs, FIG. 1). Reaction mixtures were purified by anion-exchange chromatography to remove unmodified antibodies, followed by size-exclusive chromatography (SEC) to clean-up any unreacted 18N-MCC. Subsequent analysis by reducing SDS-PAGE revealed that the heavy chain was associated with three distinct bands, which were attributed to the additions of up to three 18N strands to the available sulfhydryl sites on an IgG1 (FIG. 2, panel A). On the other hand, a single band near 30 kDa was observed, which corresponded well to the conjugation of a single 18N-MCC (6 kDa) to the light chain (23 kDa). Overall, these results confirmed the successful covalent conjugation of oligonucleotides to the HTA101 IgG.

### 2.2 Both Components of IgG-18N Retained Functional Activities

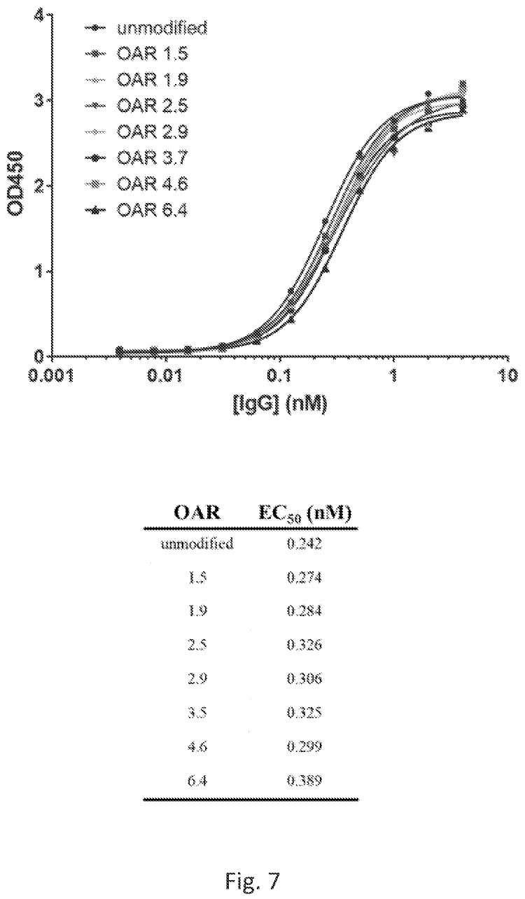

To function as a drug delivery platform, both the antibody component and the oligonucleotide component of our HTA101-18N must remain functionally uncompromised. Antigen-binding affinity, as well as the ability to hybridize stably and specifically to the complementary sequence (18NR), must not be hampered by the conjugation process. Mobility-shift assay was performed on agarose gel to assess the ability of HTA101-18N to strand-pair with its complementary sequence, 18NR-HEX, whose 5′ end was modified with a hexachlorofluorescein fluorophore (HEX) for visualization (FIG. 2, panel B). To examine the possibility of non-specific interactions between oligonucleotides and our AOC, another GC-rich 15mer sequence (15N-HEX) was included as a control. Unmodified HTA101 IgG did not interact with either fluorescent strand (lane 2 and 3), while HTA101-18N with various OARs could hybridize stoichiometrically to 18N-HEX after a brief incubation at room temperature (lane 4 to 10). Moreover, 15N-HEX did not associate with HTA101-18N at all (lane 11), indicating that association of 18N-HEX to our AOCs occurred in a strand-specific way. Relative binding affinities of HTA101-18N with various OARs towards its target antigen, HER2, were assessed by indirect ELISA (FIG. 7). Although as expected, the EC₅₀ values of modified antibodies increased slightly, overall the binding affinities of HTA101-18N, regardless of their OARs, were still at a level very similar to the unmodified IgG, confirming that the conjugation method did not cost the antibody its ability to bind strongly to the target antigen. There was also the possibility that the slight decreases in EC₅₀ values could in fact reflect, or partially result from, the somewhat hindered interactions between the Fc domain of HTA101-18N and the secondary antibody used in ELISA, rather than true affinity losses. Together, these results suggested that both the targeting and the carrying component of our AOCs remained functional, therefore meeting the prerequisites to serve as a drug delivery platform through strand-hybridization.

### 2.3 Internalization of HTA101-18N Carrying Complementary Strands into HER2-Overexpressing Cells

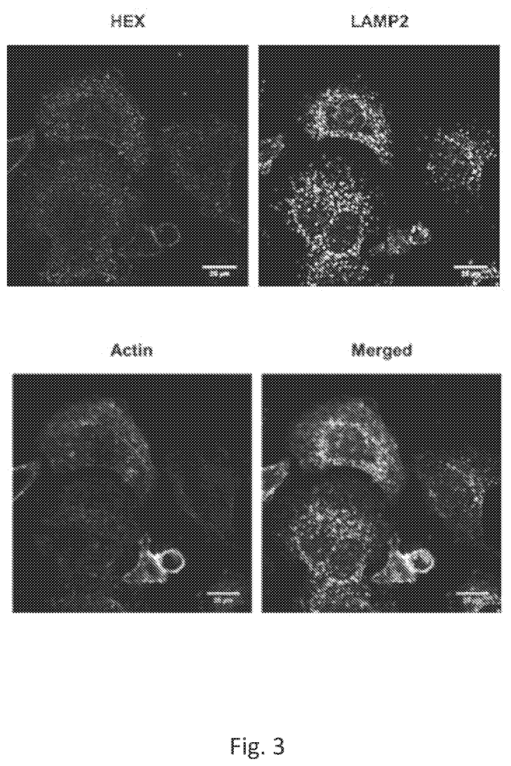

An investigation of the AOCs, carrying their complementary passenger strands, could be internalized efficiently into HER2-overexpressing cells to release their payloads against cytosolic targets. HTA101-18N (OAR 4.7) was pre-incubated with 18NR-HEX to form a double-stranded complex, which was subsequently used to treat SK-BR-3 breast cancer cells. Fluorescence of HEX allowed us to examine the spatial distribution of the passenger strand using confocal microscopy (FIG. 3). Clathrin-dependent receptor-mediated endocytosis is the primary mechanism by which ADCs enter the cells.²⁸ The actin cytoskeleton plays essential roles during the endocytic process in mammalian cells, such as scission of the cellular membrane and movement of vesicles freed from the membrane.′ Co-staining with fluorophore-conjugated phalloidin indicated that the intracellular distribution of 18NR-HEX passenger strand matched closely with that of the actin cytoskeleton. Furthermore, co-staining of LAMP2 revealed that 18NR-HEX did not co-localize with lysosomes, which was in agreement with the fact that HER2 was continuously recycled between cell surface and early endosomes without entering the lysosomal pathway.′ Together, these data suggested that 18NR-HEX was being up-taken through the HER2 endocytosis pathway. Since the HEX fluorophore was attached non-covalently to the antibody through DNA duplex formation, AOC-aided uptake would only occur if the entire double-stranded complex remained intact throughout the course of antigen-binding and receptor-mediated endocytosis. These results confirmed the viability of utilizing strand-pairing for cargo attachment and delivery, setting the stage for further testing of our AOC as a flexible drug-delivery platform.

### 2.4 HTA101-18N Paired with Toxin-bearing Complementary Strands Demonstrated Great In Vitro Potency and Selectivity Against HER2-Overexpressing Cancer Cells

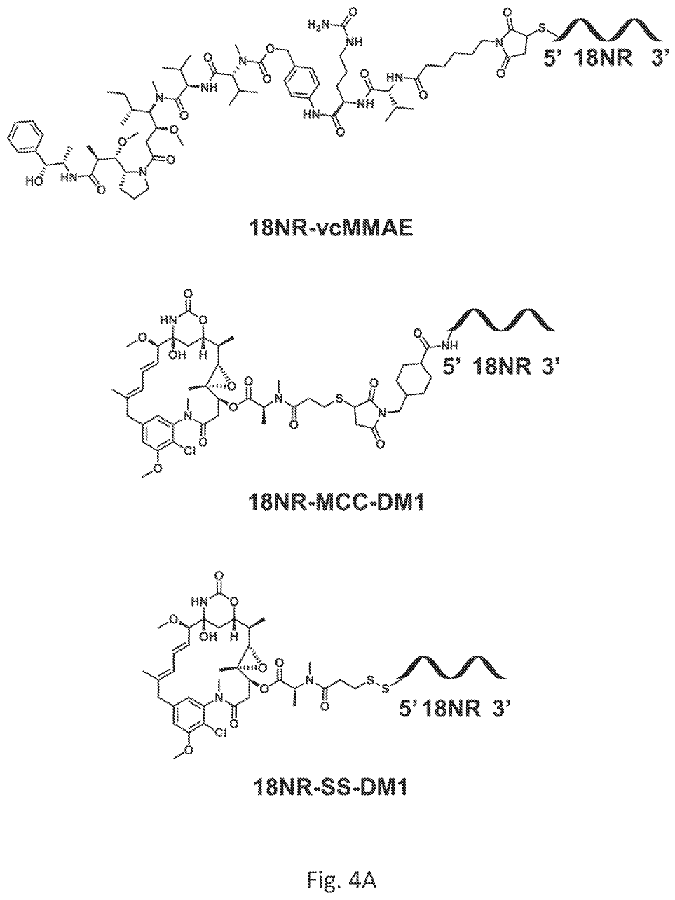

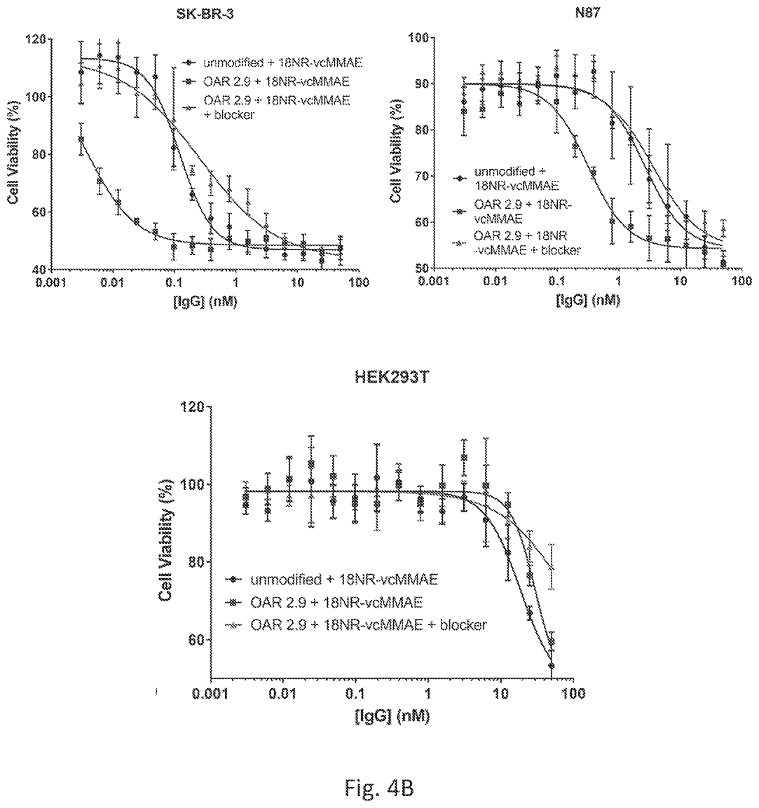

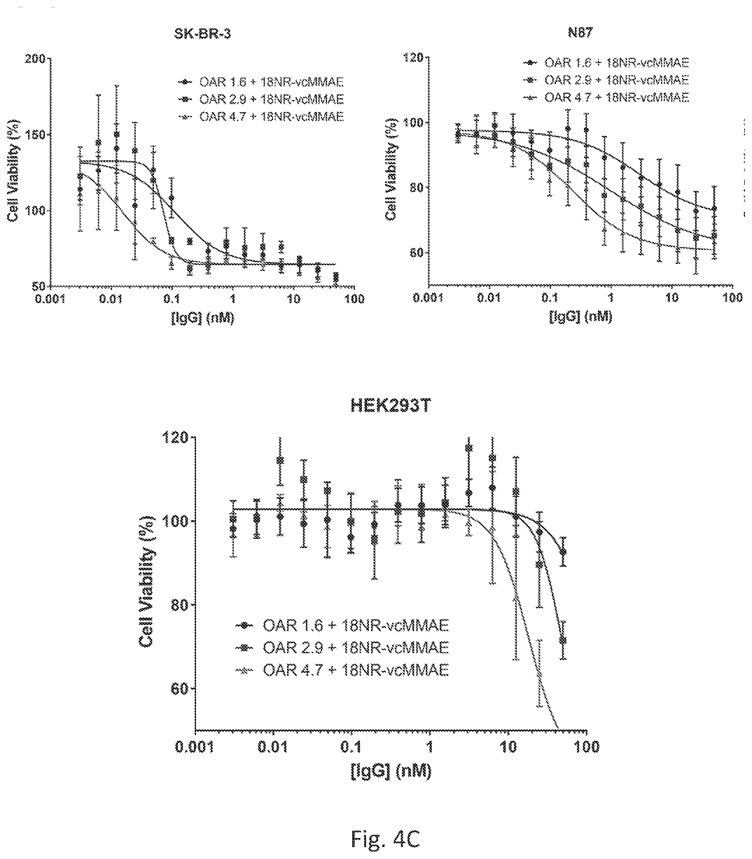

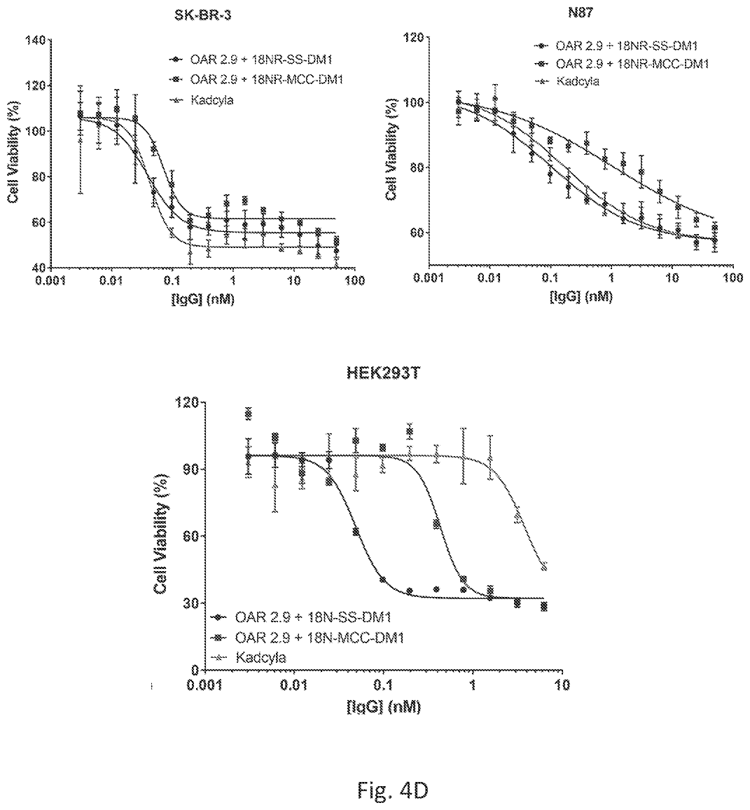

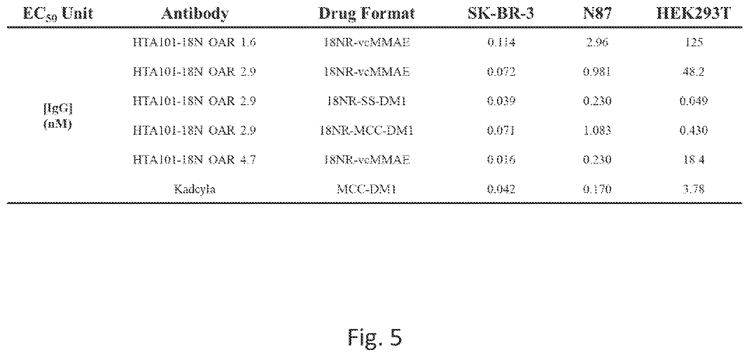

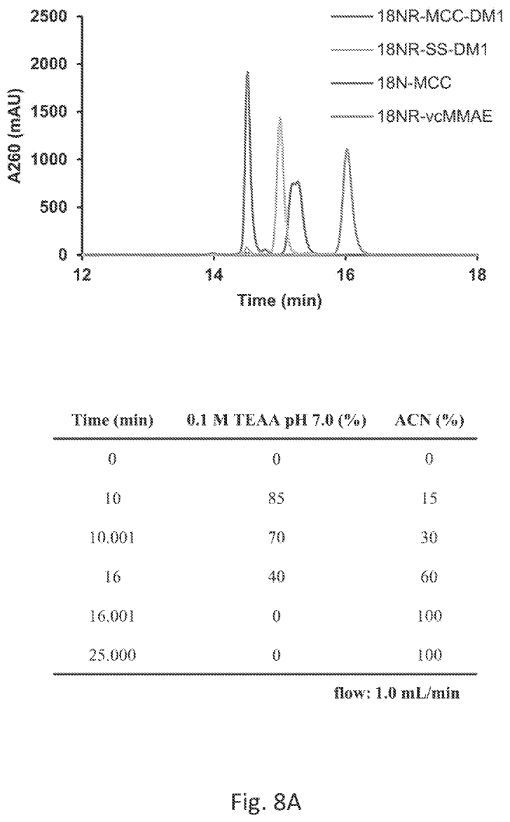

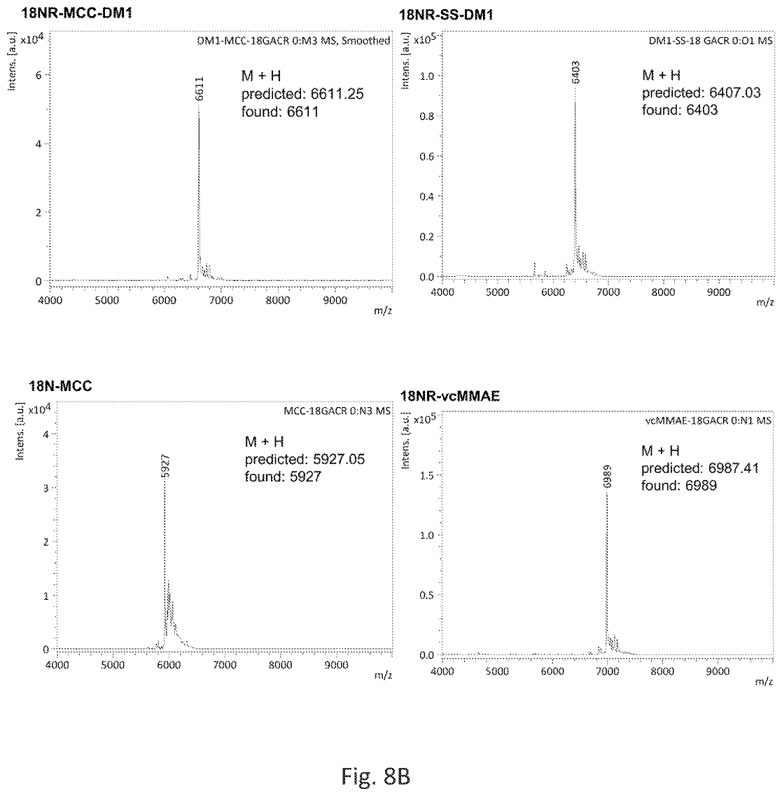

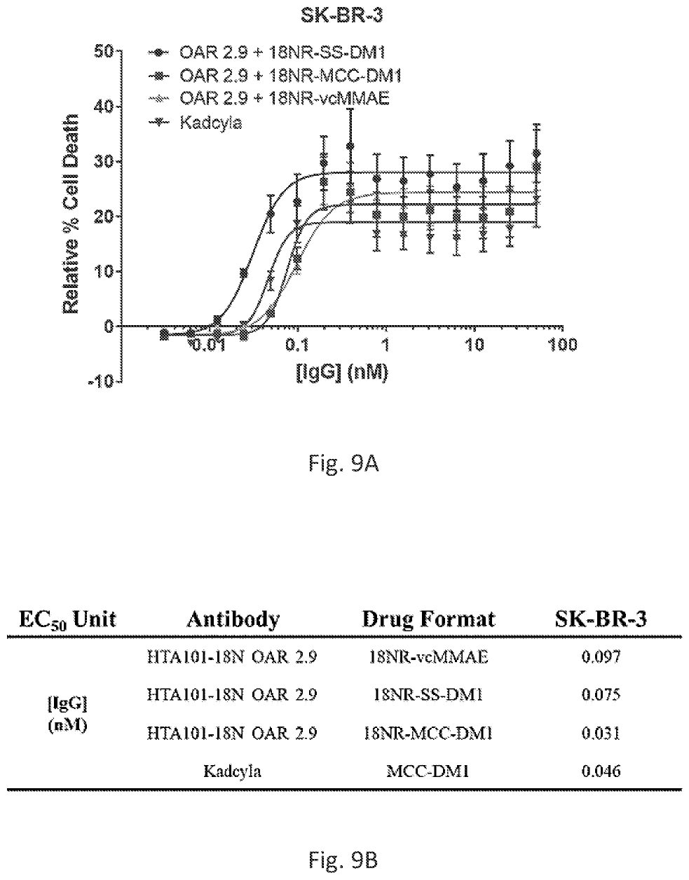

As a proof-of-concept, an in vitro WST-1 cell viability assay was performed to evaluate the potency of HTA101-18N AOC paired with drug-bearing complementary strands against HER2-overexpressing cancer cells. The results were summarized in FIG. 5. Highly potent cytotoxic drugs that are common cargos for traditional ADCs, including monomethyl auristatin E (MMAE) or mertansine (DM1), were covalently conjugated to the 5′ end of the complementary strand (18NR) through various linker formats (FIG. 4, panel A and FIG. 6). Commercially available MMAE with a protease-cleavable valine-citrulline spacer (vcMMAE) and a reactive maleimide group could be attached to 18NR complementary strand with sulfhydryl modifications in a single step (18NR-vcMMAE); DM1 was conjugated to 18NR in either a noncleavable format (18NR-MCC-DM1) or a cleavable disulfide format (18NR-SS-DM1). Purified oligonucleotides were fully characterized by reverse-phase HPLC analysis and MALDI-TOF mass spectrometry (FIG. 8). To confirm that complementary strands were internalized into HER2-positive cells via hybridization with HTA101-18N, the potency of HTA101-18N paired with 18NRvcMMAE in the absence or presence of excessive 18NR competing strands without toxin modifications was evaluated (FIG. 4, panel B). While the combination of HTA101-18N and 18NR-vcMMAE showed sub-nanomolar EC₅₀ values against SK-BR-3 and N87 cell lines over-expressing HER2, an added ten-fold excess of 18NR competitors as hybridization blockers significantly diminished its activity, proving that cytotoxic cargos were internalized via strand-pairing with AOC. Importantly, control experiments of HER2-overexpressing cells treated with a physical mixture of unmodified antibody and 18N-vcMMAE showed dose-response curves that were very similar to those treated with excessive blocker, suggesting that there existed a much less efficient pathway for the uptake of 18NR-vcMMAE independent of HER2. In comparison, control cell line HEK293T without HER2 over-expression showed nearly identical dose response curves across all three combinations. The effect of OAR on the in vitro potencies of HTA101-18N/18NR-vcMMAE was investigated as well (FIG. 4, panel C). As expected, higher OARs led to increased potencies, with N87 cells being much more sensitive to the degree of drug-loading than SK-BR-3 cells. The viability of control cells HEK293T remained unaffected across a wide range of concentrations, although cytotoxicity resulting from non-specific uptake became more prominent at higher OARs, as suggested by the EC₅₀ values in FIG. 5. This data suggested that choosing an OAR properly balancing potency and potential systemic toxicity would be crucial to further development of our strategy. The modular AOC drug delivery platform was compared to the marketed ADC Kadcyla, which had DM1 as payload linked covalently at a drug-to-antibody ratio (DAR) of 3.5 (FIG. 4, panel D). In all cases, the combination of HTA101-18N with either 18NR-SS-DM1 or 18NR-MCC-DM1 displayed potencies similar to that of Kadcyla, suggesting that drug attachment to antibody through non-covalent strand pairing did not negatively impact its delivery. As expected, 18NR-SS-DM1 paired with AOCs led to greater potencies compared to its non-cleavable counterpart 18NR-MCC-DM1, as the steric hindrance of the 18-mer oligonucleotide strand probably had some effect on the accessibility of the DM1 payload. Lastly, dose-response experiment of SK-BR-3 cells evaluated by lactate dehydrogenase (LDH) assay, gave EC₅₀ values very similar to that obtained from WST-1 assays, confirming that the viability loss resulted from bona fide cell death (FIG. 9). Overall, these results suggested that oligonucleotide strand-pairing allowed the combination of HTA101-18N and 18NR-drug to efficiently and selectively deliver a variety of cytotoxic payloads to cancer cells over-expressing the HER2 antigen.

In sum, the results observed in this study demonstrated that both the targeting antibody component and the oligonucleotide component for drug attachment remained fully functional after the conjugation. Most importantly, in vitro cytotoxicity assays demonstrated that the AOC loaded with drugs through strand hybridization was essentially as potent as the marketed ADC Kadcyla, which had DM1 bonded in the traditional covalent way, thereby proving the feasibility of utilizing strand-pairing to carry drugs for internalization. This delivery platform proved to be very versatile, capable of accepting drugs with different structures and different linker format. Formation of duplexes between oligonucleotide-drugs and the AOCs disclosed herein, as evidenced by the mobility-shift assays, was rapid and sequence-specific, potentially allowing for on-site combination of AOCs and drugs right before treatment.

The invention is also characterized by the following items.
1. An oligonucleotide conjugate, comprising
(i) a first oligonucleotide conjugate comprising a first single strand oligonucleotide conjugated to a biomolecule, wherein the first single strand oligonucleotide comprises a first nucleotide sequence; and/or
(ii) a second oligonucleotide conjugate comprising a second single strand oligonucleotide conjugated to an agent, wherein the second single strand oligonucleotide comprises a second nucleotide sequence being complementary to the first nucleotide sequence;

wherein the first and second oligonucleotide conjugates form a double-strand oligonucleotide conjugate which comprises a hybridized oligonucleotide bridge region between the first nucleotide sequence and the second nucleotide sequence, whereby the biomolecule and the agent are linked together in the double-strand oligonucleotide conjugate.
2. The oligonucleotide conjugate of Item 1, wherein the first nucleotide sequence and the second nucleotide sequence individually comprise a GC rich sequence.
3. The oligonucleotide conjugate of Item 1 or 2, wherein the first nucleotide sequence and the second nucleotide sequence individually have substantially no secondary structure.
4. The oligonucleotide conjugate of any of Items 1 to 3, wherein the first nucleotide sequence and the second nucleotide sequence individually contain from about 12 nucleotides to about 80 nucleotides in length.
5. The oligonucleotide conjugate of any of Items 1 to 4, wherein the first nucleotide sequence and the second nucleotide sequence have a melting temperature (Tm) of at least 38° C.
6. The oligonucleotide conjugate of Item 5, wherein the Tm is 40° C.−70° C.
7. The oligonucleotide conjugate of any of Items 1 to 6, wherein the first single strand oligonucleotide is conjugated at 5′-end to the biomolecule, and/or the second single strand oligonucleotide is conjugated at 5′-end to the agent; or the first single strand oligonucleotide is conjugated at 3′-end to the biomolecule, and/or the second single strand oligonucleotide is conjugated at 3′-end to the agent.
8. The oligonucleotide conjugate of any one of Items 1 to 7, wherein the first single strand oligonucleotide, the second single strand oligonucleotide, or both are DNAs, RNAs, or hybrids thereof.
9. The oligonucleotide conjugate of any one of Items 1 to 8, wherein the first single strand oligonucleotide, the single strand second oligonucleotide, or both comprise at least one modified nucleotide residue.
10. The oligonucleotide conjugate of any of Items 2 to 9, wherein the GC rich sequence comprises the nucleotide sequence 5′-SSWSSWSWSSSWWSSWSS-3′ as set forth in SEQ ID NO:1, wherein each S is independently selected from G or C and each W is independently selected from A or T; or the nucleotide sequence 5′-SSWSSWWSSSWSWSSWSS-3′ as set forth in SEQ ID NO:2, wherein each S is independently selected from G or C and each W is independently selected from A or T.
11. The oligonucleotide conjugate of Item 10, wherein the GC rich sequence comprises the nucleotide sequence 5′-GGWCCWGWCCGWWGGWCC-3′ as set forth in SEQ ID NO: 3 wherein each W is independently selected from A or T; or the nucleotide sequence 5′-GGWCCWWCGGWCWGGWCC-3′ as set forth in SEQ ID NO: 4, wherein each W is independently selected from A or T.
12. The oligonucleotide conjugate of Item 11, wherein the GC rich sequence comprises

the nucleotide sequence
(SEQ ID NO: 5)
5′-GGACCAGACCGAAGGACC-3′;
or
the nucleotide sequence
(SEQ ID NO: 6)
5′-GGTCCTTCGGTCTGGTCC-3′.

13. The oligonucleotide conjugate of any of Items 1 to 12, wherein the biomolecule is a peptide, a polypeptide, a nucleic acid, or a carbohydrate molecule.
14. The oligonucleotide conjugate of any of Items 1 to 12, wherein the biomolecule is an antibody.
15. The oligonucleotide conjugate of any of Items 1 to 14, wherein the first single strand oligonucleotide is conjugated to the biomolecule to form the first oligonucleotide via a chemical linker.
16. The oligonucleotide conjugate of Item 15, wherein the chemical linker comprises a succinimide moiety, a maleimide moiety, a hydrazine moiety, a hydrazone moiety, an azide moiety, a terminal alkyne moiety, a strained terminal alkyne moiety, or a phosphine moiety.
17. The oligonucleotide conjugate of any of Items 1 to 16, wherein the molar ratio between the biomolecule and the first single strand oligonucleotide in the first oligonucleotide conjugate ranges from 1:1 to 1:6.
18. The oligonucleotide conjugate of any of Items 1 to 17, wherein the agent in the second oligonucleotide conjugate is a therapeutic agent or a diagnostic agent.
19. The oligonucleotide conjugate of Item 18, wherein the therapeutic agent is a cytotoxic agent.
20. The oligonucleotide conjugate of Item 19, wherein the cytotoxic agent is monomethyl auristatin E (MMAE) or mertansine (DM1).
21. The oligonucleotide conjugate of Item 18, wherein the diagnostic agent is a fluorescent moiety, a luminescent moiety or a radioactive moiety.
22. A method of preparing an oligonucleotide-linked molecule, the method comprising
(a) providing a first oligonucleotide conjugate comprising a first oligonucleotide conjugated to a biomolecule, wherein the first oligonucleotide comprises a first nucleotide sequence;
(b) providing a second oligonucleotide conjugate comprising a second oligonucleotide conjugated to an agent, wherein the second oligonucleotide comprises a second nucleotide sequence being complementary to the first nucleotide sequence; and
(c) incubating the first oligonucleotide conjugate and the second oligonucleotide conjugate under conditions allowing for hybridization between the first oligonucleotide and the second oligonucleotide, thereby producing an oligonucleotide-linked molecule carrying both of the biomolecule and the agent.
23. The method of Item 22, wherein the first nucleotide sequence and the second nucleotide sequence are as defined in any of Items 2 to 12.
24. The method of Item 22 or 23, wherein the biomolecule is a peptide, a polypeptide, a nucleic acid, or a carbohydrate molecule.
25. The method of any of Items 22 to 24, wherein the biomolecule is an antibody.
26. The method of any of Items 22 to 25, wherein the agent in the second oligonucleotide conjugate is a therapeutic agent or a diagnostic agent.
27. The method of Item 26, wherein the therapeutic agent is a cytotoxic agent.
28. The method of Item 27, wherein the cytotoxic agent is monomethyl auristatin E (MMAE) or mertansine (DM1).
29. The method of any of Items 22 to 28, wherein the first oligonucleotide is conjugated at 5′-end to the biomolecule, and/or the second oligonucleotide is conjugated at 5′-end to the agent; or the first oligonucleotide is conjugated at 3′-end to the biomolecule, and/or the second oligonucleotide is conjugated at 3′-end to the agent.
30. The method of any of Items 22 to 29, further comprising harvesting the oligonucleotide-linked molecule produced in step (c).
31. The method of any of Items 22 to 30, wherein step (a) is performed by a process comprising:
   (a1) adding a first functional handle to the 5′ end of the first oligonucleotide to form a reactive first oligonucleotide;
   (a2) reacting the reactive first oligonucleotide with the biomolecule to produce the first oligonucleotide conjugate.
32. The method of Item 31, wherein the first functional handle is a maleimide moiety and the biomolecule is a polypeptide comprising a free—SH group.
33. The method of Item 32, wherein step (al) is performed by reacting the first oligonucleotide with succinimidyl 4-(N-maleimidomethyl)cyclohexane-1-carboxylate.
34. The method of Item 32 or 33, wherein the polypeptide biomolecule is treated by a reducing agent to produce the free—SH group.
35. The method of any one of Items 22 to 34, wherein step (b) is performed by a process comprising
(b1) adding a second functional handle to the 5′ end of the second oligonucleotide to produce a reactive second oligonucleotide; and
(b2) incubating the reactive second oligonucleotide and the agent in the presence of a cross-linking reagent to produce the agent conjugated with the second oligonucleotide.
36. The method of Item 35, wherein the second functional handle is a —SH group or a —NH₂ group.
37. The method of Item 35 or 36 wherein the cross-linking agent is succinimidyl 4-(N-maleimidomethyl)cyclohexane-1-carboxylate or 2,2′-dithiodipyridine.
38. A method for treating or diagnosing a disease in a subject in need thereof, the method comprising administering to the subject an oligonucleotide conjugate of any of Items 1 to 21.
39. A pharmaceutical composition comprising an oligonucleotide conjugate of any of Items 1 to 21 and a pharmaceutically acceptable carrier.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

### REFERENCES

1. Abdollahpour-Alitappeh, M. et al. Antibody—Drug Conjugates (ADCs) for Cancer Therapy:Strategies, Challenges, and Successes. Journal of Cellular Physiology 234, 5628-5642 (2019).
2. Beck, A., Goetsch, L., Dumontet, C. & Corvaia, N. Strategies and Challenges for the Next Generation of Antibody—Drug Conjugates. Nature Reviews Drug Discovery 16, 315 (2017).
3. Jain, N., Smith, S. W., Ghone, S. & Tomczuk, B. Current ADC Linker Chemistry. Pharmaceutical Research 32, 3526-3540 (2015).
4. Tsuchikama, K. & An, Z. Antibody-Drug Conjugates: Recent Advances in Conjugation and Linker Chemistries. Protein & Cell 9, 33-46 (2018).
5. Kern, J. C. et al. Discovery of Pyrophosphate Diesters as Tunable, Soluble, and Bioorthogonal Linkers for Site-Specific Antibody—Drug Conjugates. Journal of the American Chemical Society 138, 1430-1445 (2016).
6. Zhao, R. Y. et al. Synthesis and Evaluation of Hydrophilic Linkers for Antibody— Maytansinoid Conjugates. Journal of Medicinal Chemistry 54, 3606-3623 (2011).
7. Dovgan, I., Koniev, 0, Kolodych, S. & Wagner, A. Antibody—Oligonucleotide Conjugates as Therapeutic, Imaging, and Detection Agents. Bioconjugate Chemistry (2019).
8. Sano, T., Smith, C. & Cantor, C. Immuno-PCR: Very Sensitive Antigen Detection by Means of Specific Antibody-DNA Conjugates. Science 258, 120-122(1992).
9. Kazane, S. A. et al. Site-specific DNA-antibody Conjugates for Specific and Sensitive Immuno-PCR. Proc Natl Acad Sci USA 109, 3731-3736 (2012).
10. van Buggenum, J. A. G. L. et al. A Covalent and Cleavable Antibody-DNA Conjugation Strategy for Sensitive Protein Detection via Immuno-PCR. Scientific Reports 6, 22675 (2016).
11. Gong, H. et al. Simple Method To Prepare Oligonucleotide-Conjugated Antibodies and Its Application in Multiplex Protein Detection in Single Cells. Bioconjugate Chemistry 27, 217-225 (2016).
12. Patra, M., Zarschler, K., Pietzsch, H.-J., Stephan, H. & Gasser, G. New Insights into the Pretargeting Approach to Image and Treat Tumours. Chemical Society Reviews 45, 6415-6431 (2016).
13. Kuijpers, W. H. A., Kaspersen, F. M., Veeneman, G. H., Van Boeckel, C. A. A. & Bos, E. S. Specific Recognition of Antibody-Oligonucleotide Conjugates by Radiolabeled Antisense Nucleotides: A Novel Approach for Two-Step Radioimmunotherapy of Cancer. Bioconjugate Chemistry 4, 94-102 (1993).
14. Bos, E. S. et al. Evaluation of DNA-DNA Hybridization as a Two-Step Approach in Radioimmunotherapy of Cancer. Cancer Research 54, 3479 (1994).
15. Liu, G. et al. A Preclinical 188Re Tumor Therapeutic Investigation Using MORF/cMORF Pretargeting and an AntiTAG-72 Antibody CC49. Cancer Biology & Therapy 10, 767-774 (2010).
16. Liu, G. et al. Tumor Pretargeting in Mice Using 99mTc-Labeled Morpholino, a DNA Analog. Journal of Nuclear Medicine 43, 384-391 (2002).
17. Walker, I., Irwin, W. J. & Akhtar, S. Improved Cellular Delivery of Antisense Oligonucleotides Using Transferrin Receptor Antibody-Oligonucleotide Conjugates. Pharmaceutical Research 12, 1548-1553 (1995).
18. Cuellar, T. L. et al. Systematic Evaluation of Antibody-Mediated siRNA Delivery Using an Industrial Platform of THIOMAB—siRNA Conjugates. Nucleic Acids Research 43, 1189-1203 (2014).
19. Jones, D. S. et al. Conjugates of Double-Stranded Oligonucleotides with Poly(ethylene glycol) and Keyhole Limpet Hemocyanin: A model for Treating Systemic Lupus Erythematosus. Bioconjugate Chemistry 5, 390-399 (1994).
20. Sun, H. et al. Oligonucleotide Aptamers: New Tools for Targeted Cancer Therapy. Mol Ther Nucleic Acids 3, e182-e182 (2014).
21. Loke, S. L. et al. Characterization of Oligonucleotide Transport into Living Cells. Proceedings of the National Academy of Sciences 86, 3474-3478 (1989). Akhtar, S. & Juliano, R. L. Cellular Uptake and Intracellular Fate of Antisense Oligonucleotides. Trends in Cell Biology 2, 139-144 (1992).
22. Okahata, Y et al. Kinetic Measurements of DNA Hybridization on an Oligonucleotide-Immobilized 27-MHz Quartz Crystal Microbalance. Analytical Chemistry 70, 1288-1296 (1998).
23. Zhang, J. X. et al. Predicting DNA Hybridization Kinetics from Sequence. Nature Chemistry 10, 91 (2017).
24. Lang, K. & Chin, J. W. Bioorthogonal Reactions for Labeling Proteins. ACS Chemical Biology 9, 16-20 (2014).
25. Kuo, W-Y et al. Antibody-Drug Conjugates with HER2-targeting Antibodies from Synthetic Antibody Libraries Are Highly Potent Against HER2-positive Human Gastric Tumor in Xenograft Models. mAbs 11, 153-165 (2019).
26. Chudasama, V, Maruani, A. & Caddick, S. Recent Advances in the Construction of Antibody—Drug Conjugates. Nature Chemistry 8, 114 (2016).
27. Ritchie, M., Tchistiakova, L. & Scott, N. Implications of Receptor-Mediated Endocytosis and Intracellular Trafficking Dynamics in the Development of Antibody-Drug Conjugates. MAbs 5, 13-21 (2013).
28. Galletta, B. J., Mooren, O. L. & Cooper, J. A. Actin Dynamics and Endocytosis in Yeast and Mammals. Current Opinion in Biotechnology 21, 604-610 (2010).

1. An oligonucleotide conjugate, comprising
(i) a first oligonucleotide conjugate comprising a first single strand oligonucleotide conjugated to a biomolecule, wherein the first single strand oligonucleotide comprises a first nucleotide sequence; and/or
(ii) a second oligonucleotide conjugate comprising a second single strand oligonucleotide conjugated to an agent, wherein the second single strand oligonucleotide comprises a second nucleotide sequence being complementary to the first nucleotide sequence;
wherein the first and second oligonucleotide conjugates form a double-strand oligonucleotide conjugate which comprises a hybridized oligonucleotide bridge region between the first nucleotide sequence and the second nucleotide sequence, whereby the biomolecule and the agent are linked together in the double-strand oligonucleotide conjugate.

2. The oligonucleotide conjugate of claim 1, wherein the first nucleotide sequence and the second nucleotide sequence individually comprise a GC rich sequence.

3. The oligonucleotide conjugate of claim 1, wherein the first nucleotide sequence and the second nucleotide sequence individually have substantially no secondary structure.

4. The oligonucleotide conjugate of claim 1, wherein the first nucleotide sequence and the second nucleotide sequence individually contain from about 12 nucleotides to about 80 nucleotides in length.

5. The oligonucleotide conjugate of claim 1, wherein the first nucleotide sequence and the second nucleotide sequence have a melting temperature (Tm) of at least 38° C.

6. The oligonucleotide conjugate of claim 5, wherein the Tm is 40° C.−70° C.

7. The oligonucleotide conjugate of claim 1, wherein
the first single strand oligonucleotide is conjugated at 5′-end to the biomolecule, and/or the second single strand oligonucleotide is conjugated at 5′-end to the agent; or
the first single strand oligonucleotide is conjugated at 3′-end to the biomolecule, and/or the second single strand oligonucleotide is conjugated at 3′-end to the agent.

8. The oligonucleotide conjugate of claim 1, wherein the first single strand oligonucleotide, the second single strand oligonucleotide, or both are DNAs, RNAs, or hybrids thereof.

9. The oligonucleotide conjugate of claim 1, wherein the first single strand oligonucleotide, the single strand second oligonucleotide, or both comprise at least one modified nucleotide residue.

10. The oligonucleotide conjugate of claim 2, wherein the GC rich sequence comprises
the nucleotide sequence 5′-SSWSSWSWSSSWWSSWSS-3′ as set forth in SEQ ID NO:1, wherein each S is independently selected from G or C and each W is independently selected from A or T; or
the nucleotide sequence 5′-SSWSSWWSSSWSWSSWSS-3′ as set forth in SEQ ID NO:2, wherein each S is independently selected from G or C and each W is independently selected from A or T.

11. The oligonucleotide conjugate of claim 10, wherein the GC rich sequence comprises
the nucleotide sequence 5′-GGWCCWGWCCGWWGGWCC-3′ as set forth in SEQ ID NO: 3 wherein each W is independently selected from A or T; or
the nucleotide sequence 5′-GGWCCWWCGGWCWGGWCC-3′ as set forth in SEQ ID NO: 4, wherein each W is independently selected from A or T.

12. The oligonucleotide conjugate of claim 11, wherein the GC rich sequence comprises
the nucleotide sequence
(SEQ ID NO: 5)
5′-GGACCAGACCGAAGGACC-3′;
or
the nucleotide sequence
(SEQ ID NO: 6)
5′-GGTCCTTCGGTCTGGTCC-3′.

13. The oligonucleotide conjugate of claim 1, wherein the biomolecule is a peptide, a polypeptide, a nucleic acid, or a carbohydrate molecule.

14. The oligonucleotide conjugate of claim 1, wherein the biomolecule is an antibody.

15. The oligonucleotide conjugate of claim 1, wherein the first single strand oligonucleotide is conjugated to the biomolecule to form the first oligonucleotide via a chemical linker.

16. The oligonucleotide conjugate of claim 15, wherein the chemical linker comprises a succinimide moiety, a maleimide moiety, a hydrazine moiety, a hydrazone moiety, an azide moiety, a terminal alkyne moiety, a strained terminal alkyne moiety, or a phosphine moiety.

17. The oligonucleotide conjugate of claim 1, wherein the molar ratio between the biomolecule and the first single strand oligonucleotide in the first oligonucleotide conjugate ranges from 1:1 to 1:6.

18. The oligonucleotide conjugate of claim 1, wherein the agent in the second oligonucleotide conjugate is a therapeutic agent or a diagnostic agent.

19. The oligonucleotide conjugate of claim 18, wherein the therapeutic agent is a cytotoxic agent.

20. The oligonucleotide conjugate of claim 19, wherein the cytotoxic agent is monomethyl auristatin E (MMAE) or mertansine (DM1).

21. The oligonucleotide conjugate of claim 18, wherein the diagnostic agent is a fluorescent moiety, a luminescent moiety or a radioactive moiety.

22. A method of preparing an oligonucleotide-linked molecule, the method comprising
(a) providing a first oligonucleotide conjugate comprising a first oligonucleotide conjugated to a biomolecule, wherein the first oligonucleotide comprises a first nucleotide sequence;
(b) providing a second oligonucleotide conjugate comprising a second oligonucleotide conjugated to an agent, wherein the second oligonucleotide comprises a second nucleotide sequence being complementary to the first nucleotide sequence; and
(c) incubating the first oligonucleotide conjugate and the second oligonucleotide conjugate under conditions allowing for hybridization between the first oligonucleotide and the second oligonucleotide, thereby producing an oligonucleotide-linked molecule carrying both of the biomolecule and the agent.

23. The method of claim 22, wherein the first nucleotide sequence and the second nucleotide sequence individually comprise a GC rich sequence.

24. The method of claim 2, wherein the biomolecule is a peptide, a polypeptide, a nucleic acid, or a carbohydrate molecule.

25. The method of claim 22, wherein the biomolecule is an antibody.

26. The method of claim 22, wherein the agent in the second oligonucleotide conjugate is a therapeutic agent or a diagnostic agent.

27. The method of claim 26, wherein the therapeutic agent is a cytotoxic agent.

28. The method of claim 27, wherein the cytotoxic agent is monomethyl auristatin E (MMAE) or mertansine (DM1).

29. The method of claim 22, wherein
the first oligonucleotide is conjugated at 5′-end to the biomolecule, and/or the second oligonucleotide is conjugated at 5′-end to the agent; or
the first oligonucleotide is conjugated at 3′-end to the biomolecule, and/or the second oligonucleotide is conjugated at 3′-end to the agent.

30. The method of claim 22, further comprising harvesting the oligonucleotide-linked molecule produced in step (c).

31. The method of claim 22, wherein step (a) is performed by a process comprising:
(a1) adding a first functional handle to the 5′ end of the first oligonucleotide to form a reactive first oligonucleotide;
(a2) reacting the reactive first oligonucleotide with the biomolecule to produce the first oligonucleotide conjugate.

32. The method of claim 31, wherein the first functional handle is a maleimide moiety and the biomolecule is a polypeptide comprising a free—SH group.

33. The method of claim 32, wherein step (a1) is performed by reacting the first oligonucleotide with succinimidyl 4-(N-maleimidomethyl)cyclohexane-1-carboxylate.

34. The method of claim 3, wherein the polypeptide biomolecule is treated by a reducing agent to produce the free—SH group.

35. The method of claim 22, wherein step (b) is performed by a process comprising
(b1) adding a second functional handle to the 5′ end of the second oligonucleotide to produce a reactive second oligonucleotide; and
(b2) incubating the reactive second oligonucleotide and the agent in the presence of a cross-linking reagent to produce the agent conjugated with the second oligonucleotide.

36. The method of claim 35, wherein the second functional handle is a —SH group or a —NH₂ group.

37. The method of claim 35 wherein the cross-linking agent is succinimidyl 4-(N-maleimidomethyl)cyclohexane-1-carboxylate or 2,2′-dithiodipyridine.

38. A method for treating or diagnosing a disease in a subject in need thereof, the method comprising administering to the subject an oligonucleotide conjugate of claim 1, or a pharmaceutical composition comprising such oligonucleotide conjugate.

39. A pharmaceutical composition comprising an oligonucleotide conjugate of claim 1 and a pharmaceutically acceptable carrier.


SEQUENCE LISTING (6 sequences)

SEQ ID NO: 1 - DNA, 18 bp, Artificial Sequence
  Oligonucleotide linker
sswsswswssswwsswss

SEQ ID NO: 2 - DNA, 18 bp, Artificial Sequence
  Oligonucleotide linker
sswsswwssswswsswss

SEQ ID NO: 3 - DNA, 18 bp, Artificial Sequence
  Oligonucleotide linker
ggwccwgwccgwwggwcc

SEQ ID NO: 4 - DNA, 18 bp, Artificial Sequence
  Oligonucleotide linker
ggwccwwcggwcwggwcc

SEQ ID NO: 5 - DNA, 18 bp, Artificial Sequence
  18N linker sequence
ggaccagaccgaaggacc

SEQ ID NO: 6 - DNA, 18 bp, Artificial Sequence
  18NR linker sequence
ggtccttcggtctggtcc